(19) United States
(12) Patent Application Publication
(10) Pub. No.: US 2021/0301019 A1
(43) Pub. Date: Sep. 30, 2021

(54) BI-SPECIFIC FUSION PROTEINS FOR DEPLETION OF REGULATORY T CELLS

(71) Applicant: Aetio Biotherapy, Inc., Dallas, TX (US)

(72) Inventor: Kuo-Fu Tseng, Dallas, TX (US)

(21) Appl. No.: 17/213,588

(22) Filed: Mar. 26, 2021

Related U.S. Application Data
(60) Provisional application No. 62/994,903, filed on Mar. 26, 2020.

(51) Int. Cl. C07K 16/28; C07K 14/705; A61P 35/00

(52) U.S. Cl. CPC C07K 16/2818; C07K 14/705; A61P 35/00; C07K 2317/73; C07K 2317/732; C07K 2317/734; C07K 2319/30

(57) ABSTRACT

The present invention includes fusion proteins, nucleic acids and methods of making and using a protein that binds to antigen presenting cells, such as macrophages and dendritic cells, and an antibody of binding fragment thereof that specifically binds to a regulatory T cell (Treg), wherein the fusion protein reduces the activity of the Treg.

### CROSS-REFERENCE TO RELATED APPLICATIONS

This application claims priority to U.S. Provisional Application Ser. No. 62/994,903, filed Mar. 26, 2020, the entire contents of which are incorporated herein by reference.

### TECHNICAL FIELD OF THE INVENTION

The present invention relates in general to the field of Bi-specific Fusion Proteins for Depletion of Regulatory T cells.

### STATEMENT OF FEDERALLY FUNDED RESEARCH

Not applicable.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Mar. 26, 2021, is named AEBI1001.txt and is 90,011 bytes in size.

### BACKGROUND OF THE INVENTION

Without limiting the scope of the invention, its background is described in connection with regulatory T cells.

One such patent is U.S. Pat. No. 10,329,617, issued to Drake, et al., entitled “Compositions and methods for modulating an immune response.” This invention is said to include compositions and methods for modulating an immune response. In particular embodiments, such compositions and methods modulate regulatory T cell suppressive activity by inhibiting the expression of biological activity of Helios.

Another such patent is U.S. Pat. No. 8,546,137, issued to Cannon, et al., entitled “Inhibition of dendritic cell-driven regulatory T cell activation and potentiation of tumor antigen-specific T cell responses by interleukin-15 and MAP kinase inhibitor.” These inventors are said to teach that if dendritic cells loaded with a tumor antigen are cultured in interleukin-15 (IL-15), or if T cells activated by the dendritic cells are cultured in IL-15, Treg activity that is specific for the tumor antigen is reduced. This reduction in Treg activity is said to result in an increase in anti-tumor immune response.

What is needed are novel fusion proteins that overcome the problems in the prior art, by improving the ability to specifically target intratumoral regulatory T cells.

### SUMMARY OF THE INVENTION

In one embodiment, the present invention includes a regulatory T cell (Treg) depleting bispecific polypeptide comprising: a first polypeptide comprising an antibody variable light and constant light domain; a second polypeptide comprising an antibody variable heavy domain, constant heavy domain and an Fc domain, wherein the first and second polypeptide form a first antigen-binding domain that binds a Treg cell surface antigen; and a third polypeptide comprising a macrophage or dendritic cell surface protein that specifically binds a second T cell target, wherein the first and second T cell targets are different targets, and wherein the bispecific polypeptide depletes or inactivates Treg cells. In one aspect, the first antigen-binding domain binds to CTLA4, OX40, IL2Ra, and CD25. In another aspect, the anti-CTLA4 antigen-binding domain is a pH sensitive anti-CTLA4 antibody or fragment thereof. In another aspect, the macrophage or dendritic cell surface protein is Sirpα, CD47, and FcR. In another aspect, the third polypeptide further comprises an antibody variable heavy and constant heavy domain between the Fc domain and the macrophage or dendritic cell surface protein that forms a second antigen-binding domain. In another aspect, the bispecific polypeptide further comprises a fourth polypeptide comprising an antibody variable light and constant light domain that forms an antibody variable domain with the third polypeptide. In another aspect, the first and second antigen-binding domains target the same antigen or different antigens. In another aspect, the antigen-binding domain is selected from the group consisting of: an Fv fragment, a single chain Fv fragment, a disulfide-bonded Fv fragment, an Fab fragment; an Fab′ fragment, or an F(ab)₂ fragment). In another aspect, the Fc domain is a human Fc domain or a variant of a said domain, where the domain is an IgG1, IgG2, IgG3 or IgG4 domain, preferably an IgG1 or IgG4 domain. In another aspect, the polypeptide is capable of inducing antibody dependent cell cytotoxicity (ADCC), antibody-dependent cellular phagocytosis (ADCP), complement-dependent cytotoxicity (CDC), and/or apoptosis. In another aspect, the polypeptide is capable of inducing tumor immunity by depleting Treg cells. In another aspect, the Fc domain is a mutant Fc domain that has higher antibody dependent cell cytotoxicity (ADCC), antibody-dependent cellular phagocytosis (ADCP), or complement-dependent cytotoxicity (CDC), when compared to a non-mutant Fc domain. In another aspect, the bispecific polypeptide comprises a prodrug version of the first and second polypeptides that only bind to a T cell inactivating cell surface antigen under activating conditions. In another aspect, the Fc domain is a human Fc domain or a variant Fc domain, where the Fc domain is an IgG1, IgG2, IgG3 or IgG4, IgA1, IgA2, IgE, IgD or IgM domain. In another aspect, the polypeptide does at least one of: (1) bind to intratumoral Tregs that expresses CTLA4; (2) CTLA4 blockade to allow T cell activation; (3) Treg depletion in an FcR dependent manner; (4) comprise an FcγR that induces ADCC (antibody-dependent cellular cytotoxicity) and ADCP (antibody-dependent phagocytosis); or (5) binds CD47 and promotes Treg depletion by macrophages, wherein the macrophage or dendritic cell surface protein is Sirpα. In another aspect, the protein or polypeptide comprises at least one of: an anti-hCTLA4/hSirpα-Fc heterodimer comprising a first chain that is a hSirpα-Fc, a second chain comprising anti-hCTLA4 V_{H}-C_{H}-Fc, and a third chain comprising an anti-hCTLA4 V_{L}-C_{L}; a Pro anti-hCTLA4-Fc fusion protein homodimer comprising from amino to carboxy an anti-hCTLA4 V_{L}-C_{L}-cleavable linker-V_{H}-C_{H}-Fc; a single chain Pro anti-hCTLA4-Fc fusion protein comprising from amino to carboxy an anti-hCTLA4 V_{L}-Fc-cleavable linker-V_{H}-Fc; a Pro anti-hCTLA4/hSirpα-Fc comprising two fusion protein from amino to carboxy anti-hCTLA4 V_{L}-C_{L}-cleavable linker-V_{H}-C_{H}-Fc and the second chain is an hSirpα-Fc; a Pro anti-hCTLA4/hSirpα-Fc single chain fusion protein comprising from amino to carboxy anti-hCTLA4 V_{L}-Fc-cleavable linker-V_{H}-Fc-uncleavable flexible linker-hSirpα; a Pro anti-hCTLA4/hSirpα-Fc single chain fusion protein comprising from amino to carboxy hSirpα-uncleavable flexible linker-anti-hCTLA4 V_{L}-Fc-cleavable linker-V_{H}-Fc; a Pro anti-hCTLA4/Pro hSirpα-Fc single chain fusion protein comprising from amino to carboxy anti-hCTLA4 V_{L}-C_{L}-cleavable linker-V_{H}-C_{H}-Fc-cleavable linker-hSirpα-Fc; an anti-hCTLA4/Pro hSirpα-Fc comprising two chains in which the first chain is an anti-hCTLA4 V_{L-}C_{L} and the second chain is the anti-hCTLA4 V_{H-}C_{H}-Fc-cleavable linker-hSirpα-Fc; an anti-hCTLA4/Pro hSirpα-Fc heterodimer comprising three fusion protein chains, in which the first chain is an anti-hCTLA4 V_{L}-C_{L}, the second chain is an anti-hCTLA4 V_{H-}C_{H}-Fc, and the third chain is a blocking peptide for hSirpα-cleavable linker-hSirpα-Fc; or an anti-hCTLA4/Pro hSirpα-Fc homodimer comprising two fusion protein chains, in which the first chain is an anti-hCTLA4 V_{L}-C_{L}, the second chain is an anti-hCTLA4 V_{H-}C_{H}-Fc-hSirpα-cleavable linker —a blocking peptide for hSirpα. In one aspect, the polypeptide comprises at least one of SEQ ID NOS:1 to 3; SEQ ID NO:4; SEQ ID NO:5; SEQ ID NO:6 and 1; SEQ ID NO:8; SEQ ID NO:9; SEQ ID NO:10; SEQ ID NOS:11 and 12; SEQ ID NOS:13 to 15; and/or SEQ ID NOS:16 and 17.

In another embodiment, the present invention includes a regulatory T cell (Treg) depleting protein comprising: a first polypeptide comprising an antibody variable light and constant light domain; a second polypeptide comprising an antibody variable heavy domain, constant heavy domain, an Fc domain and a macrophage or dendritic cell surface protein, wherein the first and second polypeptide form a first antigen-binding domain that binds a Treg cell surface antigen, and wherein the macrophage or dendritic cell surface protein specifically binds a second T cell target, and wherein the first and second T cell targets are different targets, and wherein the bispecific polypeptide depletes or inactivates Treg cells. In one aspect, the macrophage or dendritic cell surface protein is at an amino, a carboxy, or both the amino and carboxy end of the second polypeptide. In another aspect, the macrophage or dendritic cell surface protein is at an amino, a carboxy, or both the amino and carboxy end of the first polypeptide. In another aspect, the macrophage or dendritic cell surface protein is at an amino, a carboxy, or both the amino and carboxy end of the first and the second polypeptide. In another aspect, the antigen-binding domain binds to CTLA4, OX40, IL2Ra, and CD25. In another aspect, the anti-CTLA4 antigen-binding domain is a pH sensitive anti-CTLA4 antibody or fragment thereof. In another aspect, the macrophage or dendritic cell surface protein is Sirpα, CD47, or FcR. In another aspect, the antigen-binding domain is selected from the group consisting of: an Fv fragment, a single chain Fv fragment, a disulfide-bonded Fv fragment, an Fab fragment; an Fab′ fragment, or an F(ab)₂ fragment). In another aspect, the Fc domain is a human Fc domain or a variant of a said domain, where the domain is an IgG1, IgG2, IgG3 or IgG4 domain, preferably an IgG1 or IgG4 domain. In another aspect, the protein is capable of inducing antibody dependent cell cytotoxicity (ADCC), antibody-dependent cellular phagocytosis (ADCP), complement-dependent cytotoxicity (CDC), and/or apoptosis. In another aspect, the protein is capable of inducing tumor immunity by depleting Treg cells. In another aspect, the Fc domain is a mutant Fc domain that has higher antibody dependent cell cytotoxicity (ADCC), antibody-dependent cellular phagocytosis (ADCP), or complement-dependent cytotoxicity (CDC), when compared to a non-mutant Fc domain. In another aspect, the Fc domain is a human Fc domain or a variant Fc domain, where the Fc domain is an IgG1, IgG2, IgG3 or IgG4, IgA1, IgA2, IgE, IgD or IgM domain. In another aspect, the protein at least one of: (1) binds to intratumoral Tregs that expresses CTLA4; (2) blockades CTLA4 to allow T cell activation; (3) depletes Tregs in an FcR dependent manner; (4) comprises an FcγR that induces ADCC (antibody-dependent cellular cytotoxicity) and ADCP (antibody-dependent phagocytosis); or (5) wherein the macrophage or dendritic cell surface protein is Sirpα that binds CD47 and promotes Treg depletion by macrophages. In another aspect, the protein or polypeptide comprises at least one of: an anti-hCTLA4/hSirpα-Fc heterodimer comprising a first chain that is a hSirpα-Fc, a second chain comprising anti-hCTLA4 V_{H}-C_{H}-Fc, and a third chain comprising an anti-hCTLA4 V_{L}-C_{L}; a Pro anti-hCTLA4-Fc fusion protein homodimer comprising from amino to carboxy an anti-hCTLA4 V_{L}-C_{L}-cleavable linker-V_{H}-C_{H}-Fc; a single chain Pro anti-hCTLA4-Fc fusion protein comprising from amino to carboxy an anti-hCTLA4 V_{L}-Fc-cleavable linker-V_{H}-Fc; a Pro anti-hCTLA4/hSirpα-Fc comprising two fusion protein from amino to carboxy anti-hCTLA4 V_{L}-C_{L}-cleavable linker-V_{H}-C_{H}-Fc and the second chain is an hSirpα-Fc; a Pro anti-hCTLA4/hSirpα-Fc single chain fusion protein comprising from amino to carboxy anti-hCTLA4 V_{L}-Fc-cleavable linker-V_{H}-Fc-uncleavable flexible linker-hSirpα; a Pro anti-hCTLA4/hSirpα-Fc single chain fusion protein comprising from amino to carboxy hSirpα-uncleavable flexible linker-anti-hCTLA4 V_{L}-Fc-cleavable linker-V_{H}-Fc; a Pro anti-hCTLA4/Pro hSirpα-Fc single chain fusion protein comprising from amino to carboxy anti-hCTLA4 V_{L}-C_{L}-cleavable linker-V_{H}-C_{H}-Fc-cleavable linker-hSirpα-Fc; an anti-hCTLA4/Pro hSirpα-Fc comprising two chains in which the first chain is an anti-hCTLA4 V_{L-}C_{L} and the second chain is the anti-hCTLA4 V_{H-}C_{H}-Fc-cleavable linker-hSirpα-Fc; an anti-hCTLA4/Pro hSirpα-Fc heterodimer comprising three fusion protein chains, in which the first chain is an anti-hCTLA4 V_{L}-C_{L}, the second chain is an anti-hCTLA4 V_{H}-C_{H}-Fc, and the third chain is a blocking peptide for hSirpα-cleavable linker-hSirpα-Fc; or an anti-hCTLA4/Pro hSirpα-Fc homodimer comprising two fusion protein chains, in which the first chain is an anti-hCTLA4 V_{L}-C_{L}, the second chain is an anti-hCTLA4 V_{H-}C_{H}-Fc-hSirpα-cleavable linker—a blocking peptide for hSirpα. In one aspect, the polypeptide comprises at least one of SEQ ID NOS:1 to 3; SEQ ID NO:4; SEQ ID NO:5; SEQ ID NO:6 and 1; SEQ ID NO:8; SEQ ID NO:9; SEQ ID NO:10; SEQ ID NOS:11 and 12; SEQ ID NOS:13 to 15; and/or SEQ ID NOS:16 and 17.

In another embodiment, the present invention includes a method of making a regulatory T cell (Treg) depleting bispecific polypeptide comprising: providing a first polypeptide comprising an antibody variable light and constant light domain; providing a second polypeptide comprising an antibody variable heavy domain, constant heavy domain and an Fc domain, wherein the first and second polypeptide form an antigen-binding domain that binds a T cell inactivating cell surface antigen; and providing a third polypeptide comprising a macrophage or dendritic cell surface protein that specifically binds a second T cell target, wherein the first and second T cell targets are different targets, wherein the polypeptide depletes or inactivates Treg cells. In one aspect, the antigen-binding domain binds to CTLA4, OX40, IL2Ra, and CD25. In another aspect, the anti-CTLA4 antigen-binding domain is a pH sensitive anti-CTLA4 antibody or fragment thereof. In another aspect, the macrophage or dendritic cell surface protein is Sirpα, CD47, or FcR. In another aspect, the antigen-binding domain is selected from the group consisting of: an Fv fragment, a single chain Fv fragment, a disulfide-bonded Fv fragment, an Fab fragment; an Fab′ fragment, or an F(ab)₂ fragment). In another aspect, the Fc domain is a human Fc domain or a variant of a said domain, where the domain is an IgG1, IgG2, IgG3 or IgG4 domain, preferably an IgG1 or IgG4 domain. In another aspect, the polypeptide is capable of inducing antibody dependent cell cytotoxicity (ADCC), antibody-dependent cellular phagocytosis (ADCP), complement-dependent cytotoxicity (CDC), and/or apoptosis. In another aspect, the polypeptide is capable of inducing tumor immunity by depleting Treg cells. In another aspect, the Fc domain is a mutant Fc domain that has higher antibody dependent cell cytotoxicity (ADCC), antibody-dependent cellular phagocytosis (ADCP), or complement-dependent cytotoxicity (CDC), when compared to a non-mutant Fc domain. In another aspect, the method further comprises a prodrug version of the first and second polypeptides that only bind to a T cell inactivating cell surface antigen under activating conditions. In another aspect, the Fc domain is a human Fc domain or a variant Fc domain, where the Fc domain is an IgG1, IgG2, IgG3 or IgG4, IgA1, IgA2, IgE, IgD or IgM domain. In another aspect, the protein or polypeptide comprises at least one of: an anti-hCTLA4/hSirpα-Fc heterodimer comprising a first chain that is a hSirpα-Fc, a second chain comprising anti-hCTLA4 V_{H}-C_{H}-Fc, and a third chain comprising an anti-hCTLA4 V_{L}-C_{L}; a Pro anti-hCTLA4-Fc fusion protein homodimer comprising from amino to carboxy an anti-hCTLA4 V_{L}-C_{L}-cleavable linker-V_{H}-C_{H}-Fc; a single chain Pro anti-hCTLA4-Fc fusion protein comprising from amino to carboxy an anti-hCTLA4 V_{L}-Fc-cleavable linker-V_{H}-Fc; a Pro anti-hCTLA4/hSirpα-Fc comprising two fusion protein from amino to carboxy anti-hCTLA4 V_{L}-C_{L}-cleavable linker-V_{H}-C_{H}-Fc and the second chain is an hSirpα-Fc; a Pro anti-hCTLA4/hSirpα-Fc single chain fusion protein comprising from amino to carboxy anti-hCTLA4 V_{L}-Fc-cleavable linker-V_{H}-Fc-uncleavable flexible linker-hSirpα; a Pro anti-hCTLA4/hSirpα-Fc single chain fusion protein comprising from amino to carboxy hSirpα-uncleavable flexible linker-anti-hCTLA4 V_{L}-Fc-cleavable linker-V_{H}-Fc; a Pro anti-hCTLA4/Pro hSirpα-Fc single chain fusion protein comprising from amino to carboxy anti-hCTLA4 V_{L}-C_{L}-cleavable linker-V_{H}-C_{H}-Fc-cleavable linker-hSirpα-Fc; an anti-hCTLA4/Pro hSirpα-Fc comprising two chains in which the first chain is an anti-hCTLA4 V_{L-}C_{L} and the second chain is the anti-hCTLA4 V_{H-}C_{H}-Fc-cleavable linker-hSirpα-Fc; an anti-hCTLA4/Pro hSirpα-Fc heterodimer comprising three fusion protein chains, in which the first chain is an anti-hCTLA4 V_{L}-C_{L}, the second chain is an anti-hCTLA4 V_{H-}C_{H}-Fc, and the third chain is a blocking peptide for hSirpα-cleavable linker-hSirpα-Fc; or an anti-hCTLA4/Pro hSirpα-Fc homodimer comprising two fusion protein chains, in which the first chain is an anti-hCTLA4 V_{L}-C_{L}, the second chain is an anti-hCTLA4 V_{H-}C_{H}-Fc-hSirpα-cleavable linker—a blocking peptide for hSirpα. In one aspect, the polypeptide comprises at least one of SEQ ID NOS:1 to 3; SEQ ID NO:4; SEQ ID NO:5; SEQ ID NO:6 and 1; SEQ ID NO:8; SEQ ID NO:9; SEQ ID NO:10; SEQ ID NOS:11 and 12; SEQ ID NOS:13 to 15; and/or SEQ ID NOS:16 and 17.

In another embodiment, the present invention includes a method of treating, alleviating a symptom of, or delaying a progression of a cancer comprising administering an effective amount of a bispecific polypeptide comprising: a first polypeptide comprising an antibody variable light and constant light domain; a second polypeptide comprising an antibody variable heavy domain, constant heavy domain and an Fc domain, wherein the first and second polypeptide form a first antigen-binding domain that binds a Treg cell surface antigen; and a third polypeptide comprising a macrophage or dendritic cell surface protein that specifically binds a second T cell target, wherein the first and second T cell targets are different targets, and wherein the bispecific polypeptide depletes or inactivates Treg cells to a subject in need thereof. In another aspect, the cancer is selected from a bladder cancer, a bone cancer, a breast cancer, a carcinoid, a cervical cancer, a colon cancer, an endometrial cancer, a glioma, a head and neck cancer, a liver cancer, a lung cancer, a lymphoma, a melanoma, an ovarian cancer, a pancreatic cancer, a prostate cancer, a renal cancer, a sarcoma, a skin cancer, a stomach cancer, a testis cancer, a thyroid cancer, a urogenital cancer, or a urothelial cancer. In another aspect, the cancer is selected from the group consisting of acute myeloid leukemia, adrenocortical carcinoma, B-cell lymphoma, bladder urothelial carcinoma, breast ductal carcinoma, breast lobular carcinoma, carcinomas of the esophagus, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin's lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma. In another aspect, the bispecific polypeptide targets at least one of: intratumoral Treg that highly expresses CTLA4, the bispecific polypeptide blockades CTLA4 to allow T cell activation, triggers FcR cell dependent depletion, induces FcγR ADCC (antibody-dependent cellular cytotoxicity) and ADCP (antibody-dependent phagocytosis); or Sirpα binds CD47 deplete Tregs by macrophages. In another aspect, the Tregs are intratumoral Tregs. In another aspect, the protein or polypeptide comprises at least one of: an anti-hCTLA4/hSirpα-Fc heterodimer comprising a first chain that is a hSirpα-Fc, a second chain comprising anti-hCTLA4 V_{H}-C_{H}-Fc, and a third chain comprising an anti-hCTLA4 V_{L}-C_{L}; a Pro anti-hCTLA4-Fc fusion protein homodimer comprising from amino to carboxy an anti-hCTLA4 V_{L}-C_{L}-cleavable linker-V_{H}-C_{H}-Fc; a single chain Pro anti-hCTLA4-Fc fusion protein comprising from amino to carboxy an anti-hCTLA4 V_{L}-Fc-cleavable linker-V_{H}-Fc; a Pro anti-hCTLA4/hSirpα-Fc comprising two fusion protein from amino to carboxy anti-hCTLA4 V_{L}-C_{L}-cleavable linker-V_{H}-C_{H}-Fc and the second chain is an hSirpα-Fc; a Pro anti-hCTLA4/hSirpα-Fc single chain fusion protein comprising from amino to carboxy anti-hCTLA4 V_{L}-Fc-cleavable linker-V_{H}-Fc-uncleavable flexible linker-hSirpα; a Pro anti-hCTLA4/hSirpα-Fc single chain fusion protein comprising from amino to carboxy hSirpα-uncleavable flexible linker-anti-hCTLA4 V_{L}-Fc-cleavable linker-V_{H}-Fc; a Pro anti-hCTLA4/Pro hSirpα-Fc single chain fusion protein comprising from amino to carboxy anti-hCTLA4 V_{L}-C_{L}-cleavable linker-V_{H}-C_{H}-Fc-cleavable linker-hSirpα-Fc; an anti-hCTLA4/Pro hSirpα-Fc comprising two chains in which the first chain is an anti-hCTLA4 V_{L-}C_{L} and the second chain is the anti-hCTLA4 V_{H-}C_{H}-Fc-cleavable linker-hSirpα-Fc; an anti-hCTLA4/Pro hSirpα-Fc heterodimer comprising three fusion protein chains, in which the first chain is an anti-hCTLA4 V_{L}-C_{L}, the second chain is an anti-hCTLA4 V_{H-}C_{H}-Fc, and the third chain is a blocking peptide for hSirpα-cleavable linker-hSirpα-Fc; or an anti-hCTLA4/Pro hSirpα-Fc homodimer comprising two fusion protein chains, in which the first chain is an anti-hCTLA4 V_{L}-C_{L}, the second chain is an anti-hCTLA4 V_{H-}C_{H}-Fc-hSirpα-cleavable linker—a blocking peptide for hSirpα. In one aspect, the polypeptide comprises at least one of SEQ ID NOS:1 to 3; SEQ ID NO:4; SEQ ID NO:5; SEQ ID NO:6 and 1; SEQ ID NO:8; SEQ ID NO:9; SEQ ID NO:10; SEQ ID NOS:11 and 12; SEQ ID NOS:13 to 15; and/or SEQ ID NOS:16 and 17.

In another embodiment, the present invention includes a method of depleting or inactivating Tregs comprising contacting an effective amount of a bispecific polypeptide comprising: a first polypeptide comprising an antibody variable light and constant light domain; a second polypeptide comprising an antibody variable heavy domain, constant heavy domain and an Fc domain, wherein the first and second polypeptide form a first antigen-binding domain that binds a Treg cell surface antigen; and a third polypeptide comprising a macrophage or dendritic cell surface protein that specifically binds a second T cell target, wherein the first and second T cell targets are different targets, and wherein the bispecific polypeptide depletes or inactivates Treg cells, to a T cell and antigen presenting cells that express Sirpα, CD47 or FcR. In another aspect, the antibody induces antibody dependent cell cytotoxicity (ADCC), antibody-dependent cellular phagocytosis (ADCP), complement-dependent cytotoxicity (CDC), and/or apoptosis of the Treg. In another aspect, the Tregs are intratumoral Tregs. In another aspect, the protein or polypeptide comprises at least one of: an anti-hCTLA4/hSirpα-Fc heterodimer comprising a first chain that is a hSirpα-Fc, a second chain comprising anti-hCTLA4 V_{H}-C_{H}-Fc, and a third chain comprising an anti-hCTLA4 V_{L}-C_{L}; a single chain Pro anti-hCTLA4-Fc fusion protein comprising from amino to carboxy an anti-hCTLA4 V_{L}-C_{L}-cleavable linker-V_{H}-C_{H}-Fc; a single chain Pro anti-hCTLA4-Fc fusion protein comprising from amino to carboxy an anti-hCTLA4 V_{L}-Fc-cleavable linker-V_{H}-Fc; a Pro anti-hCTLA4/hSirpα-Fc comprising two fusion protein from amino to carboxy anti-hCTLA4 V_{L}-Fc-cleavable linker-V_{H}-Fc and the second chain is an hSirpα-Fc; a Pro anti-hCTLA4/hSirpα-Fc single chain fusion protein comprising from amino to carboxy anti-hCTLA4 V_{L}-Fc-cleavable linker-V_{H}-Fc-uncleavable flexible linker-hSirpα; a Pro anti-hCTLA4/hSirpα-Fc single chain fusion protein comprising from amino to carboxy hSirpα-uncleavable flexible linker-anti-hCTLA4 V_{L}-Fc-cleavable linker-V_{H}-Fc; a Pro anti-hCTLA4/Pro hSirpα-Fc single chain fusion protein comprising from amino to carboxy anti-hCTLA4 V_{L}-Fc-cleavable linker-V_{H}-Fc-hSirpα-Fc; or an anti-hCTLA4/Pro hSirpα-Fc comprising two chains in which the first chain is an anti-hCTLA4 V_{L-}C_{L} and the second chain is the anti-hCTLA4 V_{H-}C_{H}-Fc-cleavable linker-hSirpα-Fc. In one aspect, the polypeptide comprises at least one of SEQ ID NOS:1 to 3; SEQ ID NO:4; SEQ ID NO:5; SEQ ID NO:6 and 1; SEQ ID NO:8; SEQ ID NO:9; SEQ ID NO:10; SEQ ID NOS:11 and 12; SEQ ID NOS:13 to 15; and/or SEQ ID NOS:16 and 17.

In another embodiment, the present invention includes a method of making a regulatory T cell (Treg) depleting protein comprising: providing an antibody that blocks a T cell inactivating cell surface antigen, wherein the antibody comprises at least one antigen-binding domain and an Fc domain; and providing a macrophage or dendritic cell surface protein that specifically binds a second T cell target attached to at least one Fc domain polypeptide of the antibody, wherein the polypeptide depletes or inactivates Treg cells. In one aspect, the antigen-binding domain binds to CTLA4, OX40, IL2Ra, and CD25. In another aspect, the macrophage or dendritic cell surface protein is Sirpα, CD47, and FcR. In another aspect, the anti-CTLA4 antigen-binding domain is a pH sensitive anti-CTLA4 antibody or fragment thereof. In another aspect, the antigen-binding domain is selected from the group consisting of: an Fv fragment, a single chain Fv fragment, a disulfide-bonded Fv fragment, an Fab fragment; an Fab′ fragment, or an F(ab)₂ fragment). In another aspect, the Fc domain is a human Fc domain or a variant of a said domain, where the Fc domain is an IgG1, IgG2, IgG3 or IgG4 domain, preferably an IgG1 or IgG4 domain. In another aspect, the regulatory T cell (Treg) depleting protein is capable of inducing antibody dependent cell cytotoxicity (ADCC), antibody-dependent cellular phagocytosis (ADCP), complement-dependent cytotoxicity (CDC), and/or apoptosis. In another aspect, the regulatory T cell (Treg) depleting protein is capable of inducing tumor immunity by depleting Treg cells. In another aspect, the Fc domain is a mutant Fc domain that has higher antibody dependent cell cytotoxicity (ADCC), antibody-dependent cellular phagocytosis (ADCP), or complement-dependent cytotoxicity (CDC), when compared to a non-mutant Fc domain. In another aspect, the Fc domain is a human Fc domain or a variant Fc domain, where the Fc domain is an IgG1, IgG2, IgG3 or IgG4, IgA1, IgA2, IgE, IgD or IgM domain. In another aspect, the polypeptide at least one of: (1) binds to intratumoral Tregs that expresses CTLA4; (2) blockades CTLA4 to allow T cell activation; (3) depletes Tregs in an FcR dependent manner; (4) comprises an FcγR that induces ADCC (antibody-dependent cellular cytotoxicity) and ADCP (antibody-dependent phagocytosis); or (5) wherein the macrophage or dendritic cell surface protein is Sirpα that blocks CD47 and promoted Treg depletion by macrophages. In another aspect, the protein or polypeptide comprises at least one of: an anti-hCTLA4/hSirpα-Fc heterodimer comprising a first chain that is a hSirpα-Fc, a second chain comprising anti-hCTLA4 V_{H}-C_{H}-Fc, and a third chain comprising an anti-hCTLA4 V_{L}-C_{L}; a Pro anti-hCTLA4-Fc fusion protein homodimer comprising from amino to carboxy an anti-hCTLA4 V_{L}-C_{L}-cleavable linker-V_{H}-C_{H}-Fc; a single chain Pro anti-hCTLA4-Fc fusion protein comprising from amino to carboxy an anti-hCTLA4 V_{L}-Fc-cleavable linker-V_{H}-Fc; a Pro anti-hCTLA4/hSirpα-Fc comprising two fusion protein from amino to carboxy anti-hCTLA4 V_{L}-C_{L}-cleavable linker-V_{H}-C_{H}-Fc and the second chain is an hSirpα-Fc; a Pro anti-hCTLA4/hSirpα-Fc single chain fusion protein comprising from amino to carboxy anti-hCTLA4 V_{L}-Fc-cleavable linker-V_{H}-Fc-uncleavable flexible linker-hSirpα; a Pro anti-hCTLA4/hSirpα-Fc single chain fusion protein comprising from amino to carboxy hSirpα-uncleavable flexible linker-anti-hCTLA4 V_{L}-Fc-cleavable linker-V_{H}-Fc; a Pro anti-hCTLA4/Pro hSirpα-Fc single chain fusion protein comprising from amino to carboxy anti-hCTLA4 V_{L}-C_{L}-cleavable linker-V_{H}-C_{H}-Fc-cleavable linker-hSirpα-Fc; an anti-hCTLA4/Pro hSirpα-Fc comprising two chains in which the first chain is an anti-hCTLA4 V_{L-}C_{L} and the second chain is the anti-hCTLA4 V_{H-}C_{H}-Fc-cleavable linker-hSirpα-Fc; an anti-hCTLA4/Pro hSirpα-Fc heterodimer comprising three fusion protein chains, in which the first chain is an anti-hCTLA4 V_{L}-C_{L}, the second chain is an anti-hCTLA4 V_{H-}C_{H}-Fc, and the third chain is a blocking peptide for hSirpα-cleavable linker-hSirpα-Fc; or an anti-hCTLA4/Pro hSirpα-Fc homodimer comprising two fusion protein chains, in which the first chain is an anti-hCTLA4 V_{L}-C_{L}, the second chain is an anti-hCTLA4 V_{H-}C_{H}-Fc-hSirpα-cleavable linker—a blocking peptide for hSirpα. In one aspect, the polypeptide comprises at least one of SEQ ID NOS:1 to 3; SEQ ID NO:4; SEQ ID NO:5; SEQ ID NO:6 and 1; SEQ ID NO:8; SEQ ID NO:9; SEQ ID NO:10; SEQ ID NOS:11 and 12; SEQ ID NOS:13 to 15; and/or SEQ ID NOS:16 and 17.

In another embodiment, the present invention includes a method of treating, alleviating a symptom of, or delaying a progression of a cancer comprising administering an effective amount of a protein comprising: a first polypeptide comprising an antibody variable light and constant light domain; a second polypeptide comprising an antibody variable heavy domain, constant heavy domain, an Fc domain and a macrophage or dendritic cell surface protein, wherein the first and second polypeptide form a first antigen-binding domain that binds a Treg cell surface antigen, and wherein the macrophage or dendritic cell surface protein specifically binds a second T cell target, and wherein the first and second T cell targets are different targets, and wherein the bispecific polypeptide depletes or inactivates Treg cells to a subject in need thereof. In another aspect, the cancer is selected from a bladder cancer, a bone cancer, a breast cancer, a carcinoid, a cervical cancer, a colon cancer, an endometrial cancer, a glioma, a head and neck cancer, a liver cancer, a lung cancer, a lymphoma, a melanoma, an ovarian cancer, a pancreatic cancer, a prostate cancer, a renal cancer, a sarcoma, a skin cancer, a stomach cancer, a testis cancer, a thyroid cancer, a urogenital cancer, or a urothelial cancer. In another aspect, the cancer is selected from the group consisting of acute myeloid leukemia, adrenocortical carcinoma, B-cell lymphoma, bladder urothelial carcinoma, breast ductal carcinoma, breast lobular carcinoma, carcinomas of the esophagus, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin's lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma. In another aspect, the protein targets at least one of: intratumoral Treg that highly expresses CTLA4, the bispecific polypeptide blockades CTLA4 to allow T cell activation, triggers FcR cell dependent depletion, induces FcγR ADCC (antibody-dependent cellular cytotoxicity) and ADCP (antibody-dependent phagocytosis); or the Sirpα blocks CD47 deplete Tregs by macrophages. In another aspect, the Tregs are intratumoral Tregs. In another aspect, the protein or polypeptide comprises at least one of: an anti-hCTLA4/hSirpα-Fc heterodimer comprising a first chain that is a hSirpα-Fc, a second chain comprising anti-hCTLA4 V_{H}-C_{H}-Fc, and a third chain comprising an anti-hCTLA4 V_{L}-C_{L}; a Pro anti-hCTLA4-Fc fusion protein homodimer comprising from amino to carboxy an anti-hCTLA4 V_{L}-C_{L}-cleavable linker-V_{H}-C_{H}-Fc; a single chain Pro anti-hCTLA4-Fc fusion protein comprising from amino to carboxy an anti-hCTLA4 V_{L}-Fc-cleavable linker-V_{H}-Fc; a Pro anti-hCTLA4/hSirpα-Fc comprising two fusion protein from amino to carboxy anti-hCTLA4 V_{L}-C_{L}-cleavable linker-V_{H}-C_{H}-Fc and the second chain is an hSirpα-Fc; a Pro anti-hCTLA4/hSirpα-Fc single chain fusion protein comprising from amino to carboxy anti-hCTLA4 V_{L}-Fc-cleavable linker-V_{H}-Fc-uncleavable flexible linker-hSirpα; a Pro anti-hCTLA4/hSirpα-Fc single chain fusion protein comprising from amino to carboxy hSirpα-uncleavable flexible linker-anti-hCTLA4 V_{L}-Fc-cleavable linker-V_{H}-Fc; a Pro anti-hCTLA4/Pro hSirpα-Fc single chain fusion protein comprising from amino to carboxy anti-hCTLA4 V_{L}-C_{L}-cleavable linker-V_{H}-C_{H}-Fc-cleavable linker-hSirpα-Fc; an anti-hCTLA4/Pro hSirpα-Fc comprising two chains in which the first chain is an anti-hCTLA4 V_{L-}C_{L} and the second chain is the anti-hCTLA4 V_{H-}C_{H}-Fc-cleavable linker-hSirpα-Fc; an anti-hCTLA4/Pro hSirpα-Fc heterodimer comprising three fusion protein chains, in which the first chain is an anti-hCTLA4 V_{L}-C_{L}, the second chain is an anti-hCTLA4 V_{H-}C_{H}-Fc, and the third chain is a blocking peptide for hSirpα-cleavable linker-hSirpα-Fc; or an anti-hCTLA4/Pro hSirpα-Fc homodimer comprising two fusion protein chains, in which the first chain is an anti-hCTLA4 V_{L}-C_{L}, the second chain is an anti-hCTLA4 V_{H-}C_{H}-Fc-hSirpα-cleavable linker—a blocking peptide for hSirpα. In one aspect, the polypeptide comprises at least one of SEQ ID NOS:1 to 3; SEQ ID NO:4; SEQ ID NO:5; SEQ ID NO:6 and 1; SEQ ID NO:8; SEQ ID NO:9; SEQ ID NO:10; SEQ ID NOS:11 and 12; SEQ ID NOS:13 to 15; and/or SEQ ID NOS:16 and 17.

In another embodiment, the present invention includes a method of depleting or inactivating Tregs comprising contacting an effective amount of a bispecific polypeptide protein comprising: a first polypeptide comprising an antibody variable light and constant light domain; a second polypeptide comprising an antibody variable heavy domain, constant heavy domain, an Fc domain and a macrophage or dendritic cell surface protein, wherein the first and second polypeptide form a first antigen-binding domain that binds a Treg cell surface antigen, and wherein the macrophage or dendritic cell surface protein specifically binds a second T cell target, and wherein the first and second T cell targets are different targets, and wherein the bispecific polypeptide depletes or inactivates Treg cells to T cells and antigen presenting cells that express Sirpα, CD47 or FcR. In one aspect, the antibody induces antibody dependent cell cytotoxicity (ADCC), antibody-dependent cellular phagocytosis (ADCP), complement-dependent cytotoxicity (CDC), and/or apoptosis of the Treg. In another aspect, the Tregs are intratumoral Tregs. In another aspect, the protein or polypeptide comprises at least one of: an anti-hCTLA4/hSirpα-Fc heterodimer comprising a first chain that is a hSirpα-Fc, a second chain comprising anti-hCTLA4 V_{H}-C_{H}-Fc, and a third chain comprising an anti-hCTLA4 V_{L}-C_{L}; a Pro anti-hCTLA4-Fc fusion protein homodimer comprising from amino to carboxy an anti-hCTLA4 V_{L}-C_{L}-cleavable linker-V_{H}-C_{H}-Fc; a single chain Pro anti-hCTLA4-Fc fusion protein comprising from amino to carboxy an anti-hCTLA4 V_{L}-Fc-cleavable linker-V_{H}-Fc; a Pro anti-hCTLA4/hSirpα-Fc comprising two fusion protein from amino to carboxy anti-hCTLA4 V_{L}-C_{L}-cleavable linker-V_{H}-C_{H}-Fc and the second chain is an hSirpα-Fc; a Pro anti-hCTLA4/hSirpα-Fc single chain fusion protein comprising from amino to carboxy anti-hCTLA4 V_{L}-Fc-cleavable linker-V_{H}-Fc-uncleavable flexible linker-hSirpα; a Pro anti-hCTLA4/hSirpα-Fc single chain fusion protein comprising from amino to carboxy hSirpα-uncleavable flexible linker-anti-hCTLA4 V_{L}-Fc-cleavable linker-V_{H}-Fc; a Pro anti-hCTLA4/Pro hSirpα-Fc single chain fusion protein comprising from amino to carboxy anti-hCTLA4 V_{L}-C_{L}-cleavable linker-V_{H}-C_{H}-Fc-cleavable linker-hSirpα-Fc; an anti-hCTLA4/Pro hSirpα-Fc comprising two chains in which the first chain is an anti-hCTLA4 V_{L-}C_{L} and the second chain is the anti-hCTLA4 V_{H-}C_{H}-Fc-cleavable linker-hSirpα-Fc; an anti-hCTLA4/Pro hSirpα-Fc heterodimer comprising three fusion protein chains, in which the first chain is an anti-hCTLA4 V_{L}-C_{L}, the second chain is an anti-hCTLA4 V_{H-}C_{H}-Fc, and the third chain is a blocking peptide for hSirpα-cleavable linker-hSirpα-Fc; or an anti-hCTLA4/Pro hSirpα-Fc homodimer comprising two fusion protein chains, in which the first chain is an anti-hCTLA4 V_{L}-C_{L}, the second chain is an anti-hCTLA4 V_{H-}C_{H}-Fc-hSirpα-cleavable linker—a blocking peptide for hSirpα. In one aspect, the polypeptide comprises at least one of SEQ ID NOS:1 to 3; SEQ ID NO:4; SEQ ID NO:5; SEQ ID NO:6 and 1; SEQ ID NO:8; SEQ ID NO:9; SEQ ID NO:10; SEQ ID NOS:11 and 12; SEQ ID NOS:13 to 15; and/or SEQ ID NOS:16 and 17.

### BRIEF DESCRIPTION OF THE DRAWINGS

For a more complete understanding of the features and advantages of the present invention, reference is now made to the detailed description of the invention along with the accompanying figures and in which:

FIGS. 1A to 1E show schematic diagrams of Treg-depleting recombinant proteins. FIG. 1A shows an anti-hCTLA4/hSirpα-Fc heterodimer. FIG. 1B shows two versions of a Pro anti-hCTLA4-Fc. FIG. 1C shows three versions of a Pro anti-hCTLA4/hSirpα-Fc. FIG. 1D shows a Pro anti-hCTLA4/Pro hSirpα-Fc. FIG. 1E shows three examples of anti-hCTLA4/Pro hSirpα-Fc.

FIGS. 2A and 2B are graphs in which bi-specific anti-CTLA4/Sirpα-Fc shows better tumor suppression than commercial anti-hCTLA4 antibody at 40 ug (FIG. 2A) and 10 ug (FIG. 2B).

FIGS. 3A, 3B show that Pro anti-hCTLA4 recombinant proteins show mmp14-dependent binding with hCTLA4-expressing Jurkat cells in flow cytometry-based assay. The Pro anti-hCTLA4 in 3A is in the type 2A structure in FIG. 1. The Pro anti-hCTLA4 in 3B is in the type 2B structure in FIG. 1.

FIG. 4 shows that Pro anti-hCTLA4 in the type 2A structure depletes Treg in tumor but not in peripheral tissues.

FIG. 5 shows that Pro anti-hCTLA4 in the type 2A structure suppresses tumor growth.

FIGS. 6A to 6C shows the results from a construct that includes Pro CD47-cleavable linker-CV1-FC or Pro CV1-cleavable linker-CD47-Fc (FIG. 6A), wherein cleavage of the linker at a tumor site shows surface binding of the CV1-linker-CD47-FC before cleavage of the linker (FIG. 6B), and after cleavage of the linker (FIG. 6C).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

The present invention provides for the first-time novel fusion proteins that target regulatory T cells (Treg) for depletion. Elimination of the Tregs prevents tumor cells from evading the immune system.

Unlike the prior art, the present invention targets surface markers on dendritic cells and macrophages. Many antibodies have been developed against CD47 and Sirpα to prevent their interaction and thereby prevent the suppression of myeloid cells. The present invention contains Sirpα, which is a protein expressed on macrophages and dendritic cells that initiates a signaling cascade to inhibit phagocytosis upon interaction with CD47. CD47 is a transmembrane protein expressed ubiquitously and can be upregulated in certain tumors.

As used herein, the term “activatable antibodies”, “aAb”, “pro-antibody”, or “probody” refers to a fusion protein that includes antibody antigen binding domains that are separated by a cleavable linker, such that the antibody binding domains are inoperable until the protein has been activated by the presence of the protease that cleaves the linker. The first fusion protein can be co-expressed with a second fusion protein that targets a second antigen, while the first fusion protein binds a first antigen. The first and second antigens can be the same antigen, a different antigen, or even the same antigen but bind a different epitope of the antigen. The fusion protein may also include one or more of the following: the constant light region, the constant heavy region, Fc region (wild-type or mutant), a second linker between the Fc and a second protein (e.g., a cytokine).

A nucleic acid encoding the bi-specific antibody can be part of a vector that is used to express the bi-specific antibody in a host cell, such as a bacterial, fungal, plant, or mammalian cell.

As used herein, the terms “antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain variable fragment (scFv) antibodies. An antibody other than a “bispecific” or “bifunctional” antibody is understood to have identical binding sites. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

As used herein, the term “bispecific” or “bifunctional” fusion protein is understood to have two functional domains. More commonly, the bispecific fusion protein is a bispecific antibody, which is understood to have two different antigen binding sites. For example, the bispecific antibody of the present invention will include two different antigen binding domains, e.g., a first and a second antigen binding domain that each binds a first and a second antigen, respectively. The bispecific antibody can also have two different antigen binding regions that bind the same antigen, but at two different epitopes. More commonly, the bispecific antibody will bind two different antigens. The first or second antigen will generally be a tumor specific antigen, while the other antigen binding region with bind a T cell activating molecule on a T cell.

As used herein, the term “antibody” is used in the broadest sense, and specifically covers monoclonal antibodies (including full length antibodies or other bivalent, Fc-region containing antibodies such as bivalent scFv Fc-fusion antibodies), polyclonal antibodies, multispecific antibodies (e.g., bispecific antibodies), and antibody fragments (e.g., Fab, Fab′, F(ab′)₂, Fv, scFv) so long as they exhibit the desired biological activity. Antibodies (Abs) and immunoglobulins (Igs) are glycoproteins having the same structural characteristics. While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas. The present invention includes monoclonal antibodies (and binding fragments thereof) that are completely recombinant, in other words, where the complementarity determining regions (CDRs) are genetically spliced into a human antibody backbone, often referred to as veneering an antibody. Thus, in certain aspects the monoclonal antibody is a fully synthesized antibody. In certain embodiments, the monoclonal antibodies (and binding fragments thereof) can be made in bacterial or eukaryotic cells, including plant cells.

As used herein, the term “antibody fragment” refers to a portion of a full-length antibody, generally the antigen binding or variable region and include Fab, Fab′, F(ab′)₂, Fv and scFv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments which are capable of cross-linking antigen, and a residual other fragment (which is termed pFc′). As used herein, “functional fragment” with respect to antibodies, refers to Fv, F(ab) and F(ab′)₂ fragments.

As used herein, the “Fv” fragment is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond. While the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985), relevant portions incorporated herein by reference.

As used herein, an “isolated” antibody is one that has been identified and separated and/or recovered from a component of the environment in which it was produced. Contaminant components of its production environment are materials, which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the term “antibody mutant” refers to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95, 96, 97, 98, or 99%.

As used herein, the term “variable” in the context of variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342: 877), or both, that is Chothia plus Kabat. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat et al.) The constant domains are not involved directly in binding an antibody to an antigen, but exhibit various effector function, such as participation of the antibody in antibody-dependent cellular toxicity.

The light chains of antibodies (immunoglobulin) from any vertebrate species can be assigned to one of two clearly distinct types, called kappa and lambda, based on the amino sequences of their constant domain.

Depending on the amino acid sequences of the constant domain of their heavy chains, “immunoglobulins” can be assigned to different classes. There are at least five (5) major classes of immunoglobulins: IgA, IgD, IgE, IgG and IgM, and several of these may be further divided into subclasses (isotypes), e.g., IgG-1, IgG-2, IgG-3 and IgG4; IgA-1 and IgA-2. The subunit structures and three-dimensional configurations of different classes of immunoglobulins are well known.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed and claimed invention may be made by the hybridoma method first described by Kohler and Milstein, Nature 256, 495 (1975), relevant portions incorporated herein by reference.

All monoclonal antibodies utilized in accordance with the presently disclosed and claimed invention will be either (1) the result of a deliberate immunization protocol, as described in more detail herein below; or (2) the result of an immune response that results in the production of antibodies naturally in the course of a disease or cancer.

The uses of the monoclonal antibodies of the presently disclosed and claimed invention may require administration of such or similar monoclonal antibody to a subject, such as a human. However, when the monoclonal antibodies are produced in a non-human animal, such as a rodent or chicken, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed and claimed invention can be “humanized”, that is, the antibodies are engineered such that antigenic portions thereof are removed and like portions of a human antibody are substituted therefore, while the antibodies' affinity for a specific antigen is retained. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, relevant portions incorporated herein by reference.

Humanized forms of antibodies are chimeric immunoglobulins, immunoglobulin chains or fragments thereof (such as Fab, Fab′, F(ab′)₂, Fv, scFv or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting nonhuman (i.e. rodent, chicken) CDRs or CDR sequences for the corresponding sequences of a human antibody. (See also U.S. Pat. No. 5,225,539.) In some instances, Fᵥ framework residues of the human immunoglobulin are replaced by corresponding non-human residues from the donor antibody. Humanized antibodies can also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin.

The bi-specific antibody of the present invention can also include an engineered sequence or glycosylation sites that confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

The bi-specific antibody can be a single chain variable fragment (scFv) that is a fusion of the variable regions of the heavy and light chains of immunoglobulins. This chimeric molecule retains the specificity of the original immunoglobulin, despite removal of the constant regions and the introduction of a linker peptide between the two antigen binding domains. This modification usually leaves the specificity unaltered after cleavage of the linker. These molecules were created historically to facilitate phage display where it is highly convenient to express the antigen-binding domain as a single peptide. The scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

The present invention can include the use of activatable antibodies (also referred to as pro-antibodies, or probodies) that target specific antigens on Tregs. The present invention also includes antigen binding domains that target T-cell markers. Examples of T-cell marker include CTLA4, PD-1, Lag3, S15, B7H3, B7H4, TCR-alpha, TCR-beta, and/or TIM-3. The antibodies may also bind to activating T cell markers, CD3, 41BB or OX40.

When the present invention uses an activatable antibody binding domain, it can include cleavable linkers, such as protease cleavable linkers. Examples of cleavable linker are peptides that include sequences cleaved by a tumor associated protease: MMP1, MMP2, MMP3, MMP7, MMP9, MMP 10, MMP 11, MMP 12, MMP 13, MMP 14, MMP 15, MMP 16, MMP17, MMP19, MMP20, MMP21, uPA, FAPa, or Cathepsin B. Other examples include a cleavable linker that is cleaved by proteases upregulated during apoptosis or inflammation associated responses, e.g., a caspase. Examples of caspases are Caspase 1, Caspase 2, Caspase 3, Caspase 4, Caspase 5, Caspase 6, Caspase 7, Caspase 8, Caspase 9, Caspase 10, Caspase 11, and/or Caspase 12. Unlike the activatable antibodies of the prior art, the cleavable linker of the present invention does not directly mask an antigen binding site.

The present invention can further include a cytokine, which can be separate from or included as part of a fusion protein with the bi-specific antibody, e.g., as part of the bi-specific antibody fusion protein or attached separately to the bi-specific antibody. The cytokine can be selected from at least one of: growth hormone; parathyroid hormone; thyroxine; insulin; proinsulin; relaxin; prorelaxin; glycoprotein hormones; hepatic growth factor; fibroblast growth factor; prolactin; placental lactogen; TNF-a; mullerian-inhibiting substance; gonadotropin-associated peptide; inhibin; activin; vascular endothelial growth factor; integrin; thrombopoietin (TPO); nerve growth factors; platelet-growth factor; placental growth factor, transforming growth factors (TGFs); insulin-like growth factor-1 and -11; erythropoietin (EPO); osteoinductive factors; interferons; colony stimulating factors (CSFs); lymphotoxin-alpha; lymphotoxin-beta; CD27L; CD30L; FASL; 4-1 BBL; OX40L; TRAIL; IL-1; IL-2; IL-3; IL-4; IL-5; IL-6; IL-7; IL-8; IL-9; IL-10; IL-11; IL-12; IL-13; IL-15; IL-18; IL-21; IL-22; IL-23; IL-33; IFN-a; IFN-b; IFN-g; IFN-g inducing factor (IGIF); bone morphogenetic protein (BMP); leukemia inhibitory factor (LIF); or kit ligand (KL).

The design and method of the fusion protein disclosed herein can be applied to all kinds of antibody without adding extra-elements into the antibody structure. Further, it was found that the short linkers that reduce immunogenicity and high production of antibody. Further, the present invention includes no repeated G4S linkers, thereby reducing the problem with aggregation of the fusion protein prior to cleavage linker. As described in US20200123227A1, an exemplary form of the noncleavable linker are:

GGGGS-substrate-GGGGS (SEQ ID NO:20)

(GGGGS)n-substrate-(GGGGS)n (SEQ ID NO:21), where n could be any number.

Non-cleavable linkers are (GGGGS)n where n could be any number (SEQ ID NO:22). Generally, GSx repeats can be used and varied in length, for example, Peptide Linker (noncleavable)

(SEQ ID NO: 23)
GGGGS.
(SEQ ID NO: 24)
GGGGSGGGGS.
(SEQ ID NO: 25)
GGGGSGGGGSGGGGS.
(SEQ ID NO: 26)
GGGGSGGGGSXGGGGSGGGGS,
where X = A or N.
(SEQ ID NO: 27)
GGGGSGGGGSXGGGGYGGGGS,
where X = 5, A or N, and Y = A or N.
(SEQ ID NO: 28)
GGGGSGGGGSGGGGSAAGGGGSGGGGSGGGGS.

There are many MMP cleavable sequences in the public domain, some of which can be cleaved by multiple specificities. Exemplary substrate sequences include:

Exemplary Protease Substrates, Substrate, AA Sequence

Enzyme

MMP2, MMP9
SEQ ID NO: 29
PVGLIG
MMP14
SEQ ID NO: 30
SGRSENIRTA
MMP14
SEQ ID NO: 31
SGRSPAIFTA

### EXAMPLE 1

The novel insight is to use heterodimer of Sirp alpha (Sirpα) and CTLA4 antibody to bind CD47 directly on regulatory T cells, bringing Sirpα in close proximity with Tregs. Monomeric Sirpα prevents depleting red blood cells while monomeric CTLA cannot induce the depletion of Treg in periphery. Inside tumor tissues, Treg expresses high level of both and sensitive to such heterodimer. This is done by linking Sirpα to various regulatory T cell targeting antibodies (RTTA), such as anti-CTLA4, anti-OX40 and anti-IL-2Ra. CTLA4, OX40, GITR, and IL2Ra are highly expressed on regulatory T cells.

CTLA4 is an immunoglobulin superfamily member that transmits an inhibitory signal to T cells. CTLA4 is constitutively expressed in regulatory T cells but only upregulated in conventional T cells after activation.

Because CTLA4 is constitutively expressed on Tregs, the anti-CTLA4 portion of the bispecific antibody of the present invention preferentially brings Sirpα to Tregs. Instead of targeting CD47 on tumors, anti-CTLA4-Sirpα binds CD47 on Tregs, thereby allowing macrophages and dendritic cells to deplete Tregs.

Anti-CTLA4/Sirpα-Fc heterodimer contains a monovalent anti-CTLA4 and a single Sirpα in a molecule. This format provides advantages over other formats. The monovalent anti-CTLA4 could reduce the peripheral interaction because the affinity is weaker. The effects of anti-CTLA4 antibody is expected to concentrate in tumor because CTLA4 is highly expressed on intratumoral Treg. On the other side, the affinity of single Sirpα to CD47 is also much weaker than dimeric Sirpα. This weaker affinity reduces the interaction with CD47 expressed in other cells, for example, red blood cells. The Sirpα is expected to interact with the CD47 on the same Treg to promote Treg depletion.

Anti-CTLA4 antibodies have been developed to block the inhibitory signal mediated by CTLA4 on intratumoral T cell activation. It has been shown that anti-CTLA4 antibody prevents CTLA4 from interacting with B7 on APC, which allows CD28 to interact with B7 and to activate effector T cells for killing tumor cells. However, the clinical efficacy was not as good as expected and systematic injection caused strong side effects. In mouse model, anti-CTLA4 antibodies were shown to induce partial Treg depletion in tumor via the FcγR. The Treg depletion even plays a more significant role in the antitumor activity. Current clinical use of anti-CTLA4 has not demonstrated effective Treg depletion. A high dose of antibody is required to block immune checkpoint for enhancing anti-tumor effect but it causes severe toxicity. In certain embodiments, the anti-CTLA4 is a pH sensitive anti-CTLA4 antibody or fragment thereof.

Here, the inventors incorporated Sirpα, a macrophage or dendritic cell surface protein that interacts with CD47 and produces a “don't eat me signal” for Treg to escape from being eaten by macrophages, into anti-CTLA4 antibody to further strengthen Treg depletion. The inventors have produced a bispecific antibody containing a monovalent anti-CTLA4, a single Sirpα and FcγR. This novel antibody has performed anti-tumor activity via four aspects. First, the monovalent anti-CTLA4 targets the antibody to intratumoral Treg that highly expresses CTLA4. Second, the antibody performs CTLA4 blockade to allow T cell activation. Third, such depletion depends on FcR. FcγR induces ADCC (antibody-dependent cellular cytotoxicity) and ADCP (antibody-dependent phagocytosis). Fourth, Sirpα binds CD47 to promote Treg depletion by macrophage.

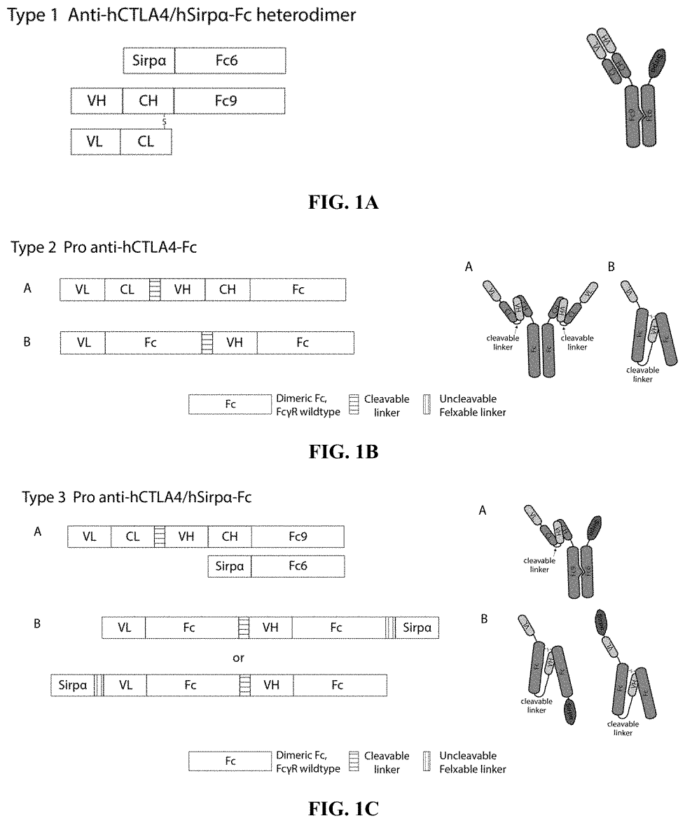

FIGS. 1A to 1E show schematic diagrams of Treg-depleting recombinant proteins. FIG. 1A shows an anti-hCTLA4/hSirpα-Fc heterodimer that includes an anti-hCTLA4 variable and constant (CH1) light and variable and constant (CH1) heavy chain connected to an Fc region (for example a wild-type Fc, or Fc variants, such as, e.g., Fc9 or Fc6). The Fc portion of the anti-hCTLA4 antibody forms a dimer (e.g., a homodimer or a heterodimer) with an Fc (for example a wild-type Fc, or Fc variants, such as Fc9 or Fc6) that is a fusion protein with hSirpα to form a hSirpα-Fc.

FIG. 1B shows two versions of a Pro anti-hCTLA4-Fc, in which a cleavable linker is carboxy between the constant light chain and the variable domain of the heavy chain, wherein cleavage of the cleavable linker causes unfolding of the Pro anti-hCTLA4-Fc to form an active (or binding) anti-hCTLA4-Fc. Alternatively, a single chain fusion protein includes from amino to carboxy anti-hCTLA4 V_{L}-Fc-cleavable linker-V_{H}-Fc.

FIG. 1C shows three versions of a Pro anti-hCTLA4/hSirpα-Fc. In the first example, a Pro anti-hCTLA4/hSirpα-Fc includes two chains, the first is an anti-hCTLA4 antibody that includes: from amino to carboxy anti-hCTLA4 V_{L}-Fc-cleavable linker-V_{H}-Fc, wherein the Fc can be a wild-type Fc, or Fc variants, such as, e.g., Fc9 or Fc6; and the second chain is an hSirpα-Fc, in which the Fc can be a wild-type Fc, or Fc variants, such as, e.g., Fc9 or Fc6. Another example is a single chain fusion protein that is from amino to carboxy anti-hCTLA4 V_{L}-Fc-cleavable linker-V_{H}-Fc-uncleavable flexible linker-hSirpα. A third option is a single chain fusion protein that is from amino to carboxy hSirpα-uncleavable flexible linker-anti-hCTLA4 V_{L}-Fc-cleavable linker-V_{H}-Fc.

FIG. 1D shows a Pro anti-hCTLA4/Pro hSirpα-Fc. In this example, a single chain fusion protein is from amino to carboxy anti-hCTLA4 V_{L}-Fc-cleavable linker-V_{H}-Fc-hSirpα-Fc.

FIG. 1E shows three examples of anti-hCTLA4/Pro hSirpα-Fc heterodimer. In the example, two chains form the anti-hCTLA4/Pro hSirpα-Fc heterodimer, in which the first chain is an anti-hCTLA4 V_{L-}C_{L} and the second chain is the anti-hCTLA4 V_{H-}C_{H}-Fc-cleavable linker-hSirpα-Fc. In the second example, three fusion proteins are included, the first fusion protein is an anti-hCTLA4 V_{L}-C_{L}, the second fusion protein is an anti-hCTLA4 V_{H-}C_{H}-Fc, and the third fusion protein is a blocking peptide for hSirpα-cleavable linker-hSirpα-Fc (in this example shown with a variant Fc, but can also be made with a wild-type Fc). In the third example, two fusion protein including a first fusion protein that is an anti-hCTLA4 V_{L}-C_{L}, and the second fusion protein is an anti-hCTLA4 V_{H-}C_{H}-Fc-hSirpα-cleavable linker-hSirpα blocking peptide.

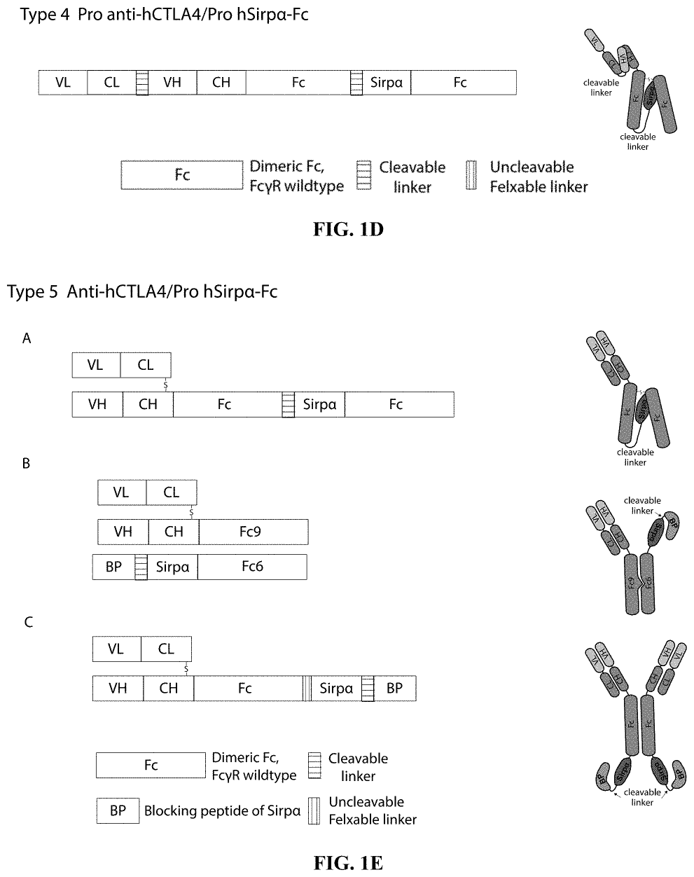

FIGS. 2A and 2B are graphs in which bi-specific anti-CTLA4/Sirpα-Fc shows better tumor suppression than commercial anti-hCTLA4 antibody at 40 ug (FIG. 2A) and 10 ug (FIG. 2B).

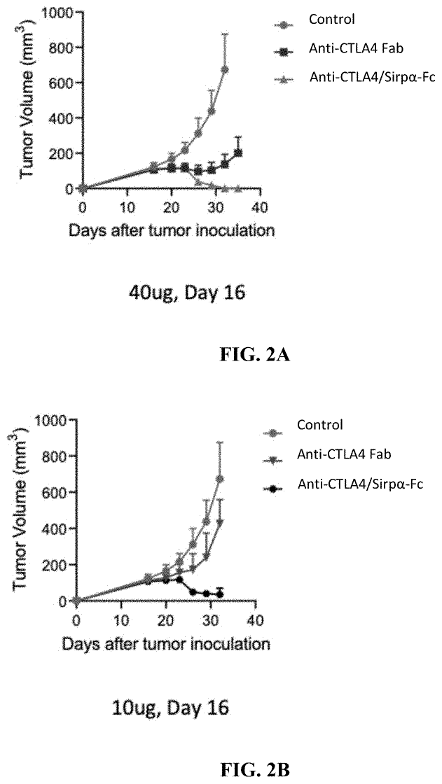

FIG. 3 shows that Pro anti-hCTLA4 recombinant proteins show mmp14-dependent binding with hCTLA4-expressing Jurkat cells in flow cytometry-based assay. The Pro anti-hCTLA4 in 3A is in the type 2A structure in FIG. 1. The Pro anti-hCTLA4 in 3B is in the type 2B structure in FIG. 1.

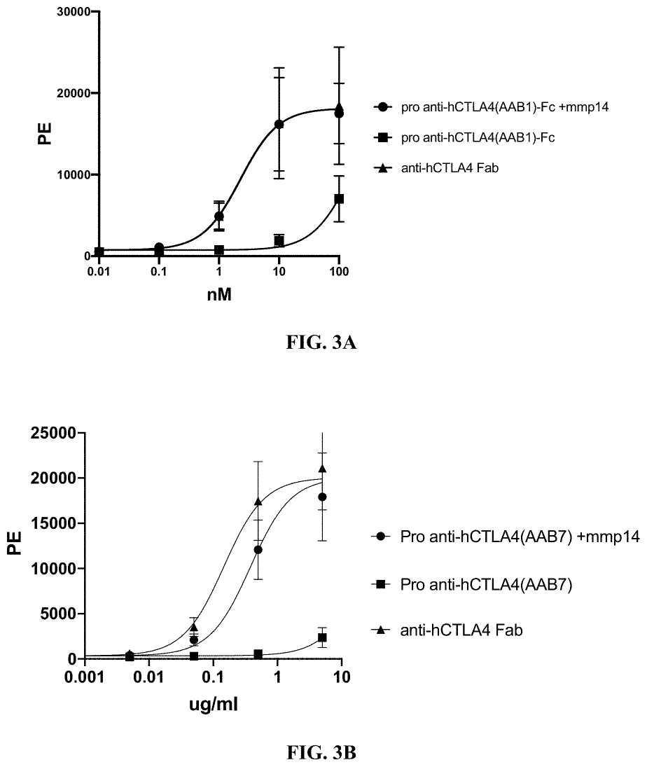

FIG. 4 shows that Pro anti-hCTLA4 in the type 2A structure depletes Treg in tumor but not in peripheral tissues.

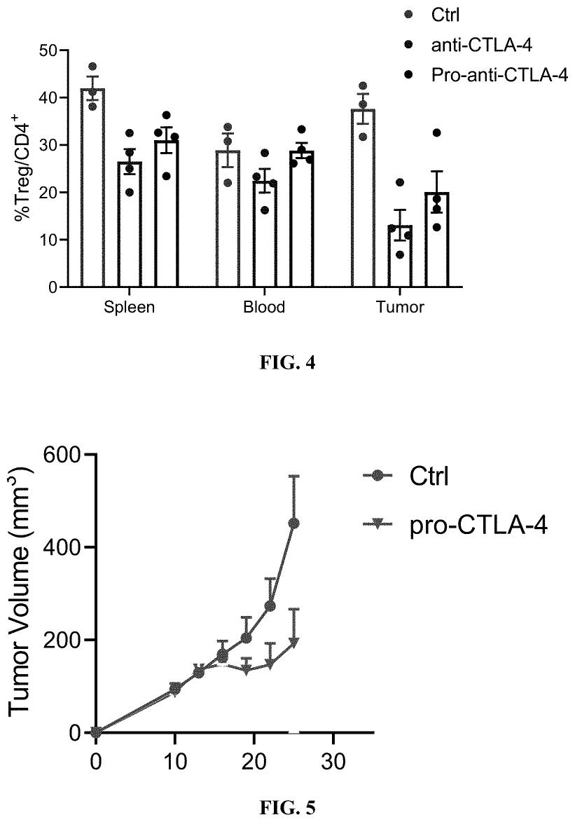

FIG. 5 shows that Pro anti-hCTLA4 in the type 2A structure suppresses tumor growth.

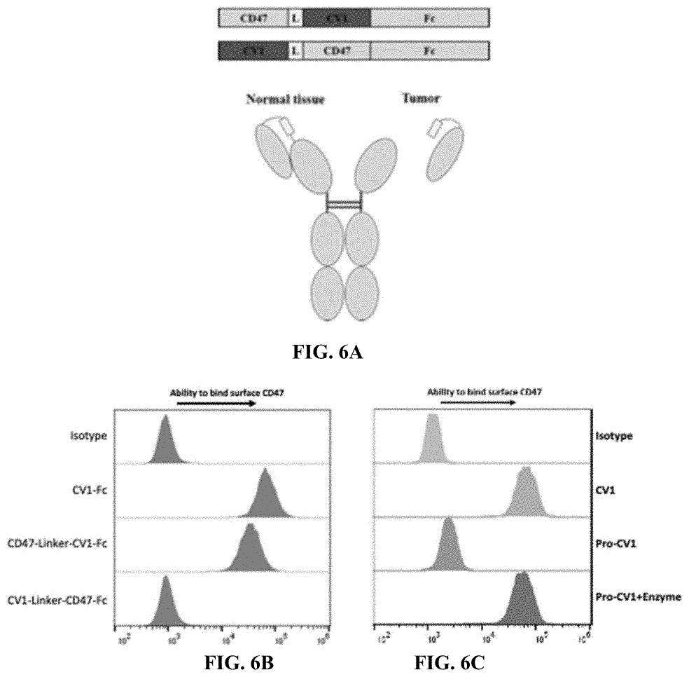

FIGS. 6A to 6C shows the results from a construct that includes Pro CD47-cleavable linker-CV1-FC or Pro CV1-cleavable linker-CD47-Fc (FIG. 6A), wherein cleavage of the linker at a tumor site shows surface binding of the CV1-linker-CD47-FC before cleavage of the linker (FIG. 6B), and after cleavage of the linker (FIG. 6C).

Type 1

The Light chain includes a signal peptide from residue 1-20, anti-CTLA4 V_{L} from residue 21 to 128 and a C_{L} from residue 129 to 235—SEQ ID NO:1

METDTLLLWVLLLWVPGSTGEIVLTQSPGTLSLSPGERATLSCRASQSVG
SSYLAWYQQKPGQAPRLLIYGAFSRATGIPDRFSGSGSGTDFTLTISRLE
PEDFAVYYCQQYGSSPWTFGQGTKVEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGEC

The Heavy chain 1 includes a signal peptide from residue 1 to 20, a V_{H} from residue 21 to 138, a C_{H} from residue 139 to 241 and a Fc9 from residue 242 to 468.—SEQ ID NO:2

METDTLLLWVLLLWVPGSTGQVQLVESGGGVVQPGRSLRLSCAASGFTFS
SYTMHWVRQAPGKGLEWVTFISYDGNNKYYADSVKGRFTISRDNSKNTLY
LQMNSLRAEDTAIYYCARTGWLGPFDYWGQGTLVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPCRDELTKNQVSLWCLVKGFYPSDIAV
EWESNGQPENNYKTTPPVLDSDGSFFLYSALTVDKSRWQQGNVFSCSVMH
EALHNHYTQKSLSLSPGK

The Heavy chain 2 includes a signal peptide from residue 1 to 20, a hSirpα from residue 21 to 140, a GGGGS linker from residue 141 to 145 and a Fc6 from residue 146 to 377.—SEQ ID NO:3

METDTLLLWVLLLWVPGSTGMEEELQVIQPDKSVLVAAGETATLRCTATS
LIPVGPIQWFRGAGPGRELIYNQKEGHFPRVTTVSDLTKRNNMDFSIRIG
NITPADAGTYYCVKFRKGSPDDVEFKSGAGTELSVRAKPSGGGGSEPKSS
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVCTLPPSRDELTKNQVSLSCAVK
GEYPSDIAVEWESNGQPENNYKTTPPVLDSDGSFKLVSKLTVDKSRWQQG
NVFSCSVMHEALHNHYTQKSLSLSPGK

Type 2A

Type 2A includes a signal peptide from residue 1 to 20, a V_{L} from residue 21 to 128, a C_{L} from residue 129 to 235, a Cleavable linker from residue 236 to 250, a V_{H} from residue 251 to 368, a C_{H} from residue 369 to 471 and a Fc from residue 472 to 698.—SEQ ID NO:4

METDTLLLWVLLLWVPGSTGEIVLTQSPGTLSLSPGERATLSCRASQSVG
SSYLAWYQQKPGQAPRLLIYGAFSRATGIPDRFSGSGSGTDFTLTISRLE
PEDFAVYYCQQYGSSPWTFGQGTKVEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGECGSSGRSENIRTAGGS
QVQLVESGGGVVQPGRSLRLSCAASGFTFSSYTMHWVRQAPGKGLEWVTF
ISYDGNNKYYADSVKGRETISRDNSKNTLYLQMNSLRAEDTAIYYCARTG
WLGPFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLEPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFELYSKLTVDKSRWQQGNVESCSVMHEALHNHYTQKSLSLSPGK

Type 2B

Type 2B includes a signal peptide from residue 1 to 20, a V_{L} from residue 21 to 128, a Fc from residue 129 to 355, a Cleavable linker from residue 356 to 361, a V_{H} from residue 362 to 479 and a Fc from residue 480 to 706.—SEQ ID NO:5

METDTLLLWVLLLWVPGSTGEIVLTQSPGTLSLSPGERATLSCRASQSVG
SSYLAWYQQKPGQAPRLLIYGAFSRATGIPDRFSGSGSGTDFTLTISRLE
PEDFAVYYCQQYGSSPWTFGQGTKVEIKDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLS
LSPGKRSENIRQVQLVESGGGVVQPGRSLRLSCAASGFTFSSYTMHWVRQ
APGKGLEWVTFISYDGNNKYYADSVKGRFTISRDNSKNTLYLQMNSLRAE
DTAIYYCARTGWLGPFDYWGQGTLVTVSSDKTHTCPPCPAPELLGGPSVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGK

Type 3A

Type 3A Heavy chain 1 includes a signal peptide from residue 1 to 20, a V_{L} from residue 21 to 128, a C_{L} from residue 129 to 235, a Cleavable linker from residue 236 to 250, a V_{H} from residue 251 to 368, a C_{H} from residue 369 to 471 and a Fc 472 to 698.—SEQ ID NO:6

METDTLLLWVLLLWVPGSTGEIVLTQSPGTLSLSPGERATLSCRASQSVG
SSYLAWYQQKPGQAPRLLIYGAFSRATGIPDRFSGSGSGTDFTLTISRLE
PEDFAVYYCQQYGSSPWTFGQGTKVEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGECGSSGRSENIRTAGGS
QVQLVESGGGVVQPGRSLRLSCAASGFTFSSYTMHWVRQAPGKGLEWVTF
ISYDGNNKYYADSVKGRFTISRDNSKNTLYLQMNSLRAEDTAIYYCARTG
WLGPFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPCRDELTKNQVSLWCLVKGFYPSDIAVEWESNGQPENNYKTTPPVLD
SDGSFFLYSALTVDKSRWQQGNVFSCSVMHEALHNHYTQKSLSLSPGK

The type 3A Heavy chain 2 includes a signal peptide from residue 1 to 20, a hSirpα from residue 21 to 140, a GGGGS linker from residue 141 to 145 and a Fc6 from residue 146 to 377.—SEQ ID NO:7

MEEELQVIQPDKSVLVAAGETATLRCTATSLIPVGPIQWFRGAGPGRELI
YNQKEGHFPRVTTVSDLTKRNNMDFSIRIGNITPADAGTYYCVKFRKGSP
DDVEFKSGAGTELSVRAKPSGGGGSEPKSSDKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVCTLPPSRDELTKNQVSLSCAVKGFYPSDIAVEWESNGQPENN
YKTTPPVLDSDGSFKLVSKLTVDKSRWQQGNVFSCSVMHEALHNHYTQKS
LSLSPGK

Type 3B

Version 1 includes a signal peptide from residue 1 to 20, a V_{L} from residue 21 to 128, a Fc from residue 129 to 355, a Cleavable linker from residue 356 to 361, a V_{H} from residue 362 to 479, a Fc from residue 480 to 706, a GGGGS linker from residue 707 to 711 and a hSirpα from residue 712 to 831.—SEQ ID NO:8

METDTLLLWVLLLWVPGSTGEIVLTQSPGTLSLSPGERATLSCRASQSVG
SSYLAWYQQKPGQAPRLLIYGAFSRATGIPDRFSGSGSGTDFTLTISRLE
PEDFAVYYCQQYGSSPWTFGQGTKVEIKDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLS
LSPGKRSENIRQVQLVESGGGVVQPGRSLRLSCAASGETFSSYTMHWVRQ
APGKGLEWVTFISYDGNNKYYADSVKGRFTISRDNSKNTLYLQMNSLRAE
DTAIYYCARTGWLGPFDYWGQGTLVTVSSDKTHTCPPCPAPELLGGPSVF
LEPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGKGGGGSMEEELQVIQPDKSVLVAAGETATLRCTATSLIPVGPIQW
FRGAGPGRELIYNQKEGHFPRVTTVSDLTKRNNMDFSIRIGNITPADAGT
YYCVKFRKGSPDDVEFKSGAGTELSVRAKPS

Type 3B 2.

Version 2 includes a signal peptide from residue 1 to 20, a hSirpα from residue 21 to 140, a GGGGS linker from residue 141 to 145, a V_{L} from residue 146 to 253, a Fc from residue 254 to 480, a Cleavable linker from residue 481 to 486, a V_{H} from residue 487 to 604 and a Fc from residue 605 to 831—SEQ ID NO:9

METDTLLLWVLLLWVPGSTGMEEELQVIQPDKSVLVAAGETATLRCTATS
LIPVGPIQWFRGAGPGRELIYNQKEGHFPRVTTVSDLTKRNNMDFSIRIG
NITPADAGTYYCVKFRKGSPDDVEFKSGAGTELSVRAKPSGGGGSEIVLT
QSPGTLSLSPGERATLSCRASQSVGSSYLAWYQQKPGQAPRLLIYGAFSR
ATGIPDRFSGSGSGTDFTLTISRLEPEDFAVYYCQQYGSSPWTFGQGTKV
EIKDKTHTCPPCPAPELLGGPSVFLEPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGKRSENIRQVQLVESGGGVVQP
GRSLRLSCAASGFTFSSYTMHWVRQAPGKGLEWVTFISYDGNNKYYADSV
KGRFTISRDNSKNTLYLQMNSLRAEDTAIYYCARTGWLGPFDYWGQGTLV
TVSSDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSR
WQQGNVFSCSVMHEALHNHYTQKSLSLSPGK

Type 4

Type 4 includes a signal peptide from residue 1 to 20, a V_{L} from residue 21 to 128, a C_{L} from residue 129 to 235, a Cleavable linker from residue 236 to 250, a V_{H} from residue 251 to 368, a C_{H} from residue 369 to 471, a Fc from residue 472 to 698, a Cleavable linker from residue 699 to 708, a hSirpα from residue 709 to 828, a GGGGS linker from residue 829 to 833 and a Fc from residue 834 to 1060.—SEQ ID NO:10

METDTLLLWVLLLWVPGSTGEIVLTQSPGTLSLSPGERATLSCRASQSVG
SSYLAWYQQKPGQAPRLLIYGAFSRATGIPDRFSGSGSGTDFTLTISRLE
PEDFAVYYCQQYGSSPWTFGQGTKVEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGECGSSGRSENIRTAGGS
QVQLVESGGGVVQPGRSLRLSCAASGFTFSSYTMHWVRQAPGKGLEWVTF
ISYDGNNKYYADSVKGRFTISRDNSKNTLYLQMNSLRAEDTAIYYCARTG
WLGPFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGKSG
RSENIRTAMEEELQVIQPDKSVLVAAGETATLRCTATSLIPVGPIQWFRG
AGPGRELIYNQKEGHFPRVTTVSDLTKRNNMDFSIRIGNITPADAGTYYC
VKFRKGSPDDVEFKSGAGTELSVRAKPSGGGGSDKTHTCPPCPAPELLGG
PSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYT
QKSLSLSPGK

Type 5A

Type 5A Light chain includes a signal peptide from residue 1 to 20, a V_{L} from residue 21 to 128 and a C_{L} from residue 129 to 235.—SEQ ID NO:11

METDTLLLWVLLLWVPGSTGEIVLTQSPGTLSLSPGERATLSCRASQSVG
SSYLAWYQQKPGQAPRLLIYGAFSRATGIPDRFSGSGSGTDFTLTISRLE
PEDFAVYYCQQYGSSPWTFGQGTKVEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGEC

Type 5A Heavy chain includes a signal peptide from residue 1 to 20, a V_{H} from residue 21 to 138, a C_{H} from residue 139 to 241, a Fc 242 from residue to 468, a Cleavable linker from residue 469 to 478, a hSirpα from residue 479 to 598, a GGGGS linker from residue 599 to 603 and a Fc from residue 604 to 830.—SEQ ID NO:12

METDTLLLWVLLLWVPGSTGQVQLVESGGGVVQPGRSLRLSCAASGFTFS
SYTMHWVRQAPGKGLEWVTFISYDGNNKYYADSVKGRFTISRDNSKNTLY
LQMNSLRAEDTAIYYCARTGWLGPFDYWGQGTLVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMH
EALHNHYTQKSLSLSPGKSGRSENIRTAMEEELQVIQPDKSVLVAAGETA
TLRCTATSLIPVGPIQWFRGAGPGRELIYNQKEGHFPRVTTVSDLTKRNN
MDFSIRIGNITPADAGTYYCVKFRKGSPDDVEFKSGAGTELSVRAKPSGG
GGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

Type 5B

Type 5A Light Chain includes a signal peptide from residue 1 to 20, a V_{L} from residue 21 to 128 and a C_{L} from residue 129 to 235.—SEQ ID NO:13

METDTLLLWVLLLWVPGSTGEIVLTQSPGTLSLSPGERATLSCRASQSVG
SSYLAWYQQKPGQAPRLLIYGAFSRATGIPDRFSGSGSGTDFTLTISRLE
PEDFAVYYCQQYGSSPWTFGQGTKVEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGEC

Type 5B Heavy Chain 1 includes a signal peptide from residue 1 to 20, a V_{H} from residue 21 to 138, a C_{H} from residue 139 to 241 and a Fc9 from residue 242 to 468.—SEQ ID NO:14

METDTLLLWVLLLWVPGSTGQVQLVESGGGVVQPGRSLRLSCAASGFTFS
SYTMHWVRQAPGKGLEWVTFISYDGNNKYYADSVKGRFTISRDNSKNTLY
LQMNSLRAEDTAIYYCARTGWLGPFDYWGQGTLVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPCRDELTKNQVSLWCLVKGFYPSDIAV
EWESNGQPENNYKTTPPVLDSDGSFFLYSALTVDKSRWQQGNVFSCSVMH
EALHNHYTQKSLSLSPGK

Type 5B Heavy Chain 2 includes a signal peptide from residue 1 to 20, a hCD47 from residue 21 to141, a Cleavable linker from residue 142 to 151, a hSirpα from residue 152 to 271, a GGGGS linker from residue 272 to 276 and a Fc from residue 277 to 508.—SEQ ID NO:15

METDTLLLWVLLLWVPGSTGQLLFNKTKSVEFTFCNDTVVIPCFVTNMEA
QNTTEVYVKWKFKGRDIYTFDGALNKSTVPTDFSSAKIEVSQLLKGDASL
KMDKSDAVSHTGNYTCEVTELTREGETIIELKYRVVSWFSPSGRSENIRT
AMEEELQVIQPDKSVLVAAGETATLRCTATSLIPVGPIQWFRGAGPGREL
IYNQKEGHFPRVTTVSDLTKRNNMDFSIRIGNITPADAGTYYCVKFRKGS
PDDVEFKSGAGTELSVRAKPSGGGGSEPKSSDKTHTCPPCPAPELLGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKA
KGQPREPQVCTLPPSRDELTKNQVSLSCAVKGFYPSDIAVEWESNGQPEN
NYKTTPPVLDSDGSFKLVSKLTVDKSRWQQGNVFSCSVMHEALHNHYTQK
SLSLSPGK

Type 5C

Type 5C Light Chain includes a signal peptide from residue 1 to 20, a V_{L} from residue 21 to 128 and a C_{L} from residue 129 to 235.—SEQ ID NO:16

METDTLLLWVLLLWVPGSTGEIVLTQSPGTLSLSPGERATLSCRASQSVG
SSYLAWYQQKPGQAPRLLIYGAFSRATGIPDRFSGSGSGTDFTLTISRLE
PEDFAVYYCQQYGSSPWTFGQGTKVEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGEC

Type 5C Heavy Chain includes a signal peptide from residue 1 to 20, a V_{H} from residue 21 to 138, a C_{H} from residue 139 to 241, a Fc from residue 242 to 468, a GGGGS linker from residue 469 to 473, a hSirpα from residue 474 to 593, a Cleavable linker from residue 594 to 603 and a hCD47 from residue 604 to 724.—SEQ ID NO:17

METDTLLLWVLLLWVPGSTGQVQLVESGGGVVQPGRSLRLSCAASGFTFS
SYTMHWVRQAPGKGLEWVTFISYDGNNKYYADSVKGRFTISRDNSKNTLY
LQMNSLRAEDTAIYYCARTGWLGPFDYWGQGTLVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMH
EALHNHYTQKSLSLSPGKGGGGSMEEELQVIQPDKSVLVAAGETATLRCT
ATSLIPVGPIQWFRGAGPGRELIYNQKEGHFPRVTTVSDLTKRNNMDFSI
RIGNITPADAGTYYCVKFRKGSPDDVEFKSGAGTELSVRAKPSSGRSENI
RTAQLLFNKTKSVEFTFCNDTVVIPCFVTNMEAQNTTEVYVKWKFKGRDI
YTFDGALNKSTVPTDFSSAKIEVSQLLKGDASLKMDKSDAVSHTGNYTCE
VTELTREGETIIELKYRVVSWFSP

human CD47—SEQ ID NO:18

MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTATGLTSFVIAILVIQVIAYI
LAVVGLSLCIAACIPMHGPLLISGLSILALAQLLGLVYMKFVASNQKTIQ
PPRKAVEEPLNAFKESKGMMNDE

CV1—SEQ ID NO:19

MEEELQIIQPDKSVLVAAGETATLRCTITSLFPVGPIQWFRGAGPGRVLI
YNQRQGPFPRVTTVSDTTKRNNMDFSIRIGNITPADAGTYYCIKFRKGSP
DDVEFKSGAGTELSVRAKPS

For example, the protein or polypeptide comprises at least one of: an anti-hCTLA4/hSirpα-Fc heterodimer comprising a first chain that is a hSirpα-Fc (SEQ ID NO:3), a second chain comprising anti-hCTLA4 V_{H}-C_{H}-Fc (SEQ ID NO:2), and a third chain comprising an anti-hCTLA4 V_{L}-C_{L} (SEQ ID NO:1).

For example, a Pro anti-hCTLA4-Fc fusion protein homodimer comprising from amino to carboxy an anti-hCTLA4 V_{L}-C_{L}-cleavable linker-V_{H}-C_{H}-Fc (SEQ ID NO:4).

For example, a single chain Pro anti-hCTLA4-Fc fusion protein comprising from amino to carboxy an anti-hCTLA4 V_{L}-Fc-cleavable linker-V_{H}-Fc (SEQ ID NO:5).

For example, a Pro anti-hCTLA4/hSirpα-Fc comprising two fusion protein from amino to carboxy anti-hCTLA4 V_{L}-C_{L}-cleavable linker-V_{H}-C_{H}-Fc (SEQ ID NO:6) and the second chain is an hSirpα-Fc (SEQ ID NO:1).

For example, a Pro anti-hCTLA4/hSirpα-Fc single chain fusion protein comprising from amino to carboxy anti-hCTLA4 V_{L}-Fc-cleavable linker-V_{H}-Fc-uncleavable flexible linker-hSirpα (SEQ ID NO:8).

For example, a Pro anti-hCTLA4/hSirpα-Fc single chain fusion protein comprising from amino to carboxy hSirpα-uncleavable flexible linker-anti-hCTLA4 V_{L}-Fc-cleavable linker-V_{H}-Fc (SEQ ID NO:9).

For example, a Pro anti-hCTLA4/Pro hSirpα-Fc single chain fusion protein comprising from amino to carboxy anti-hCTLA4 V_{L}-C_{L}-cleavable linker-V_{H}-C_{H}-Fc-cleavable linker-hSirpα-Fc (SEQ ID NO:10).

For example, an anti-hCTLA4/Pro hSirpα-Fc comprising two chains in which the first chain is an anti-hCTLA4 V_{L-}C_{L} (SEQ ID NO:11) and the second chain is the anti-hCTLA4 V_{H-}C_{H}-Fc-cleavable linker-hSirpα-Fc (SEQ ID NO:12).

For example, an anti-hCTLA4/Pro hSirpα-Fc heterodimer comprising three fusion protein chains, in which the first chain is an anti-hCTLA4 V_{L}-C_{L} (SEQ ID NO:13), the second chain is an anti-hCTLA4 V_{H-}C_{H}-Fc (SEQ ID NO:14), and the third chain is a blocking peptide for hSirpα-cleavable linker-hSirpα-Fc (SEQ ID NO:15).

An anti-hCTLA4/Pro hSirpα-Fc homodimer comprising two fusion protein chains, in which the first chain is an anti-hCTLA4 V_{L}-C_{L} (SEQ ID NO:16), the second chain is an anti-hCTLA4 V_{H-}C_{H}-Fc-hSirpα-cleavable linker—a blocking peptide for hSirpα (SEQ ID NO:17).

In one aspect, the polypeptide comprises at least one of SEQ ID NOS:1 to 3; SEQ ID NO:4; SEQ ID NO:5; SEQ ID NO:6 and 1; SEQ ID NO:8; SEQ ID NO:9; SEQ ID NO:10; SEQ ID NOS:11 and 12; SEQ ID NOS:13 to 15; and/or SEQ ID NOS:16 and 17.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A regulatory T cell (Treg) depleting bispecific polypeptide comprising:
a first polypeptide comprising an antibody variable light and constant light domain;
a second polypeptide comprising an antibody variable heavy domain, constant heavy domain and an Fc domain, wherein the first and second polypeptide form a first antigen-binding domain that binds a Treg cell surface antigen; and
a third polypeptide comprising a macrophage or dendritic cell surface protein that specifically binds a second T cell target, wherein the first and second T cell targets are different targets, and wherein the bispecific polypeptide depletes or inactivates Treg cells.

2. The bispecific polypeptide of claim 1, wherein the first antigen-binding domain binds to CTLA4, OX40, IL2Ra, and CD25, or wherein the anti-CTLA4 antigen-binding domain is a pH sensitive anti-CTLA4 antibody or fragment thereof.

3. The bispecific polypeptide of claim 1, wherein the macrophage or dendritic cell surface protein is Sirpα, CD47, and FcR.

4. The bispecific polypeptide of claim 1, wherein the third polypeptide further comprises an antibody variable heavy and constant heavy domain between the Fc domain and the macrophage or dendritic cell surface protein that forms a second antigen-binding domain.

5. The bispecific polypeptide of claim 4, further comprising a fourth polypeptide comprising an antibody variable light and constant light domain that forms an antibody variable domain with the third polypeptide, or wherein the first and second antigen-binding domains target the same antigen or different antigens.

6. The bispecific polypeptide of claim 1, wherein the antigen-binding domain is selected from the group consisting of: an Fv fragment, a single chain Fv fragment, a disulfide-bonded Fv fragment, an Fab fragment; an Fab′ fragment, or an F(ab)₂ fragment); wherein the Fc domain is a mutant Fc domain that has higher antibody dependent cell cytotoxicity (ADCC), antibody-dependent cellular phagocytosis (ADCP), complement-dependent cytotoxicity (CDC), when compared to a non-mutant Fc domain; wherein the Fc domain is a human Fc domain or a variant Fc domain, where the Fc domain is an IgG1, IgG2, IgG3 or IgG4, IgA1, IgA2, IgE, IgD or IgM domain; wherein the Fc domain is a human Fc domain or a variant of a said domain, where the domain is an IgG1, IgG2, IgG3 or IgG4 domain, preferably an IgG1 or IgG4 domain; wherein the polypeptide is capable of inducing antibody dependent cell cytotoxicity (ADCC), antibody-dependent cellular phagocytosis (ADCP), complement-dependent cytotoxicity (CDC), and/or apoptosis, wherein the polypeptide is capable of inducing tumor immunity by depleting Treg cells; or wherein the polypeptide does at least one of: (1) bind to intratumoral Tregs that expresses CTLA4; (2) CTLA4 blockade to allow T cell activation; (3) Treg depletion in an FcR dependent manner; (4) comprise an FcγR that induces ADCC (antibody-dependent cellular cytotoxicity) and ADCP (antibody-dependent phagocytosis); or (5) binds CD47 and promotes Treg depletion by macrophages, wherein the macrophage or dendritic cell surface protein is Sirpα.

7. The bispecific polypeptide of claim 1, further comprising a prodrug version of the first and second polypeptides that only bind to a T cell inactivating cell surface antigen under activating conditions.

8. The bispecific polypeptide of claim 1, wherein the polypeptide comprises at least one of:
an anti-hCTLA4/hSirpα-Fc heterodimer comprising a first chain that is a hSirpα-Fc, a second chain comprising anti-hCTLA4 V_{H}-C_{H}-Fc, and a third chain comprising an anti-hCTLA4 V_{L}-C_{L};
a Pro anti-hCTLA4-Fc fusion protein homodimer comprising from amino to carboxy an anti-hCTLA4 V_{L}-C_{L}-cleavable linker-V_{H}-C_{H}-Fc;
a single chain Pro anti-hCTLA4-Fc fusion protein comprising from amino to carboxy an anti-hCTLA4 V_{L}-Fc-cleavable linker-V_{H}-Fc;
a Pro anti-hCTLA4/hSirpα-Fc comprising two fusion protein from amino to carboxy anti-hCTLA4 V_{L}-C_{L}-cleavable linker-V_{H}-C_{H}-Fc and the second chain is an hSirpα-Fc;
a Pro anti-hCTLA4/hSirpα-Fc single chain fusion protein comprising from amino to carboxy anti-hCTLA4 V_{L}-Fc-cleavable linker-V_{H}-Fc-uncleavable flexible linker-hSirpα;
a Pro anti-hCTLA4/hSirpα-Fc single chain fusion protein comprising from amino to carboxy hSirpα-uncleavable flexible linker-anti-hCTLA4 V_{L}-Fc-cleavable linker-V_{H}-Fc;
a Pro anti-hCTLA4/Pro hSirpα-Fc single chain fusion protein comprising from amino to carboxy anti-hCTLA4 V_{L}-C_{L}-cleavable linker-V_{H}-C_{H}-Fc-cleavable linker-hSirpα-Fc;
an anti-hCTLA4/Pro hSirpα-Fc comprising two chains in which the first chain is an anti-hCTLA4 V_{L-}C_{L} and the second chain is the anti-hCTLA4 V_{H-}C_{H}-Fc-cleavable linker-hSirpα-Fc;
an anti-hCTLA4/Pro hSirpα-Fc heterodimer comprising three fusion protein chains, in which the first chain is an anti-hCTLA4 V_{L}-C_{L}, the second chain is an anti-hCTLA4 V_{H-}C_{H}-Fc, and the third chain is a blocking peptide for hSirpα-cleavable linker-hSirpα-Fc.
an anti-hCTLA4/Pro hSirpα-Fc homodimer comprising two fusion protein chains, in which the first chain is an anti-hCTLA4 V_{L}-C_{L}, the second chain is an anti-hCTLA4 V_{H-}C_{H}-Fc-hSirpα-cleavable linker—a blocking peptide for hSirpα.

9. The bispecific polypeptide of claim 1, wherein the polypeptide comprises at least one of SEQ ID NOS:1 to 3; SEQ ID NO:4; SEQ ID NO:5; SEQ ID NO:6 and 1; SEQ ID NO:8; SEQ ID NO:9; SEQ ID NO:10; SEQ ID NOS:11 and 12; SEQ ID NOS:13 to 15; SEQ ID NOS:16 and 17.

10. A regulatory T cell (Treg) depleting protein comprising:
a first polypeptide comprising an antibody variable light and constant light domain;
a second polypeptide comprising an antibody variable heavy domain, constant heavy domain, an Fc domain and a macrophage or dendritic cell surface protein, wherein the first and second polypeptide form a first antigen-binding domain that binds a Treg cell surface antigen, and wherein the macrophage or dendritic cell surface protein specifically binds a second T cell target, and wherein the first and second T cell targets are different targets, and wherein the bispecific polypeptide depletes or inactivates Treg cells.

11. The protein of claim 10, wherein the macrophage or dendritic cell surface protein is at an amino, a carboxy, or both the amino and carboxy end of the second polypeptide; wherein the macrophage or dendritic cell surface protein is at an amino, a carboxy, or both the amino and carboxy end of the first polypeptide; wherein the macrophage or dendritic cell surface protein is at an amino, a carboxy, or both the amino and carboxy end of the first and the second polypeptide; wherein the antigen-binding domain binds to CTLA4, OX40, IL2Ra, and CD25; wherein the anti-CTLA4 antigen-binding domain is a pH sensitive anti-CTLA4 antibody or fragment thereof; or wherein the macrophage or dendritic cell surface protein is Sirpα, CD47, or FcR.

12. The protein of claim 10, wherein the antigen-binding domain is selected from the group consisting of: an Fv fragment, a single chain Fv fragment, a disulfide-bonded Fv fragment, an Fab fragment; an Fab′ fragment, or an F(ab)₂ fragment); wherein the Fc domain is a human Fc domain or a variant of a said domain, where the domain is an IgG1, IgG2, IgG3 or IgG4 domain, preferably an IgG1 or IgG4 domain; wherein the Fc domain is a mutant Fc domain that has higher antibody dependent cell cytotoxicity (ADCC), antibody-dependent cellular phagocytosis (ADCP), or complement-dependent cytotoxicity (CDC), when compared to a non-mutant Fc domain; wherein the regulatory T cell (Treg) depleting protein is capable of at least one of: inducing antibody dependent cell cytotoxicity (ADCC), antibody-dependent cellular phagocytosis (ADCP), complement-dependent cytotoxicity (CDC), or apoptosis; wherein the regulatory T cell (Treg) depleting protein is capable of inducing tumor immunity by depleting Treg cells; or wherein the Fc domain is a human Fc domain or a variant Fc domain, where the Fc domain is an IgG1, IgG2, IgG3 or IgG4, IgA1, IgA2, IgE, IgD or IgM domain; or wherein the protein at least one of: (1) binds to intratumoral Tregs that expresses CTLA4; (2) blockades CTLA4 to allow T cell activation; (3) depletes Tregs in an FcR dependent manner; (4) comprises an FcγR that induces ADCC (antibody-dependent cellular cytotoxicity) and ADCP (antibody-dependent phagocytosis); or (5) wherein the macrophage or dendritic cell surface protein is Sirpα that binds CD47 and promotes Treg depletion by macrophages.

13. The protein of claim 10, wherein the protein comprises at least one of:
an anti-hCTLA4/hSirpα-Fc heterodimer comprising a first chain that is a hSirpα-Fc, a second chain comprising anti-hCTLA4 V_{H}-C_{H}-Fc, and a third chain comprising an anti-hCTLA4 V_{L}-C_{L};
a Pro anti-hCTLA4-Fc fusion protein homodimer comprising from amino to carboxy an anti-hCTLA4 V_{L}-C_{L}-cleavable linker-V_{H}-C_{H}-Fc;
a single chain Pro anti-hCTLA4-Fc fusion protein comprising from amino to carboxy an anti-hCTLA4 V_{L}-Fc-cleavable linker-V_{H}-Fc;
a Pro anti-hCTLA4/hSirpα-Fc comprising two fusion protein from amino to carboxy anti-hCTLA4 V_{L}-C_{L}-cleavable linker-V_{H}-C_{H}-Fc and the second chain is an hSirpα-Fc;
a Pro anti-hCTLA4/hSirpα-Fc single chain fusion protein comprising from amino to carboxy anti-hCTLA4 V_{L}-Fc-cleavable linker-V_{H}-Fc-uncleavable flexible linker-hSirpα;
a Pro anti-hCTLA4/hSirpα-Fc single chain fusion protein comprising from amino to carboxy hSirpα-uncleavable flexible linker-anti-hCTLA4 V_{L}-Fc-cleavable linker-V_{H}-Fc;
a Pro anti-hCTLA4/Pro hSirpα-Fc single chain fusion protein comprising from amino to carboxy anti-hCTLA4 V_{L}-C_{L}-cleavable linker-V_{H}-C_{H}-Fc-cleavable linker-hSirpα-Fc;
an anti-hCTLA4/Pro hSirpα-Fc comprising two chains in which the first chain is an anti-hCTLA4 V_{L-}C_{L} and the second chain is the anti-hCTLA4 V_{H-}C_{H}-Fc-cleavable linker-hSirpα-Fc;
an anti-hCTLA4/Pro hSirpα-Fc heterodimer comprising three fusion protein chains, in which the first chain is an anti-hCTLA4 V_{L}-C_{L}, the second chain is an anti-hCTLA4 V_{H-}C_{H}-Fc, and the third chain is a blocking peptide for hSirpα-cleavable linker-hSirpα-Fc.
an anti-hCTLA4/Pro hSirpα-Fc homodimer comprising two fusion protein chains, in which the first chain is an anti-hCTLA4 V_{L}-C_{L}, the second chain is an anti-hCTLA4 V_{H-}C_{H}-Fc-hSirpα-cleavable linker—a blocking peptide for hSirpα.

14. A method of making a regulatory T cell (Treg) depleting bispecific polypeptide comprising:
providing a first portion comprising an antibody variable light and constant light domain;
providing a second portion comprising an antibody variable heavy domain, constant heavy domain and an Fc domain, wherein the first and second polypeptide form an antigen-binding domain that binds a T cell inactivating cell surface antigen; and
providing a third portion comprising a macrophage or dendritic cell surface protein that specifically binds a second T cell target, wherein the first and second T cell targets are different targets, wherein the polypeptide depletes or inactivates Treg cells.

15. The method of claim 14, wherein the antigen-binding domain binds to CTLA4, OX40, IL2Ra, and CD25; wherein the anti-CTLA4 antigen-binding domain is a pH sensitive anti-CTLA4 antibody or fragment thereof; wherein the macrophage or dendritic cell surface protein is Sirpα, CD47, or FcR; wherein the antigen-binding domain is selected from the group consisting of: an Fv fragment, a single chain Fv fragment, a disulfide-bonded Fv fragment, an Fab fragment; an Fab′ fragment, or an F(ab)₂ fragment); wherein the Fc domain is a human Fc domain or a variant of a said domain, where the domain is an IgG1, IgG2, IgG3 or IgG4 domain, preferably an IgG1 or IgG4 domain; wherein the polypeptide is capable of inducing antibody dependent cell cytotoxicity (ADCC), antibody-dependent cellular phagocytosis (ADCP), complement-dependent cytotoxicity (CDC), or apoptosis; wherein the polypeptide is capable of inducing tumor immunity by depleting Treg cells; wherein the Fc domain is a mutant Fc domain that has higher antibody dependent cell cytotoxicity (ADCC), antibody-dependent cellular phagocytosis (ADCP), or complement-dependent cytotoxicity (CDC), when compared to a non-mutant Fc domain; or wherein the Fc domain is a human Fc domain or a variant Fc domain, where the Fc domain is an IgG1, IgG2, IgG3 or IgG4, IgA1, IgA2, IgE, IgD or IgM domain.

16. The method of claim 14, further comprising a prodrug version of the first and second polypeptides that only bind to a T cell inactivating cell surface antigen under activating conditions.

17. The method of claim 14, wherein the polypeptide comprises at least one of:
an anti-hCTLA4/hSirpα-Fc heterodimer comprising a first chain that is a hSirpα-Fc, a second chain comprising anti-hCTLA4 V_{H}-C_{H}-Fc, and a third chain comprising an anti-hCTLA4 V_{L}-C_{L};
a Pro anti-hCTLA4-Fc fusion protein homodimer comprising from amino to carboxy an anti-hCTLA4 V_{L}-C_{L}-cleavable linker-V_{H}-C_{H}-Fc;
a single chain Pro anti-hCTLA4-Fc fusion protein comprising from amino to carboxy an anti-hCTLA4 V_{L}-Fc-cleavable linker-V_{H}-Fc;
a Pro anti-hCTLA4/hSirpα-Fc comprising two fusion protein from amino to carboxy anti-hCTLA4 V_{L}-C_{L}-cleavable linker-V_{H}-C_{H}-Fc and the second chain is an hSirpα-Fc;
a Pro anti-hCTLA4/hSirpα-Fc single chain fusion protein comprising from amino to carboxy anti-hCTLA4 V_{L}-Fc-cleavable linker-V_{H}-Fc-uncleavable flexible linker-hSirpα;
a Pro anti-hCTLA4/hSirpα-Fc single chain fusion protein comprising from amino to carboxy hSirpα-uncleavable flexible linker-anti-hCTLA4 V_{L}-Fc-cleavable linker-V_{H}-Fc;
a Pro anti-hCTLA4/Pro hSirpα-Fc single chain fusion protein comprising from amino to carboxy anti-hCTLA4 V_{L}-C_{L}-cleavable linker-V_{H}-C_{H}-Fc-cleavable linker-hSirpα-Fc;
an anti-hCTLA4/Pro hSirpα-Fc comprising two chains in which the first chain is an anti-hCTLA4 V_{L-}C_{L} and the second chain is the anti-hCTLA4 V_{H-}C_{H}-Fc-cleavable linker-hSirpα-Fc;
an anti-hCTLA4/Pro hSirpα-Fc heterodimer comprising three fusion protein chains, in which the first chain is an anti-hCTLA4 V_{L}-C_{L}, the second chain is an anti-hCTLA4 V_{H-}C_{H}-Fc, and the third chain is a blocking peptide for hSirpα-cleavable linker-hSirpα-Fc.
an anti-hCTLA4/Pro hSirpα-Fc homodimer comprising two fusion protein chains, in which the first chain is an anti-hCTLA4 V_{L}-C_{L}, the second chain is an anti-hCTLA4 V_{H-}C_{H}-Fc-hSirpα-cleavable linker—a blocking peptide for hSirpα.

18. A method of treating, alleviating a symptom of, or delaying a progression of a cancer comprising administering an effective amount of a bispecific polypeptide of claim 1 to a subject in need thereof.

19. The method of claim 18, wherein the cancer is selected from a bladder cancer, a bone cancer, a breast cancer, a carcinoid, a cervical cancer, a colon cancer, an endometrial cancer, a glioma, a head and neck cancer, a liver cancer, a lung cancer, a lymphoma, a melanoma, an ovarian cancer, a pancreatic cancer, a prostate cancer, a renal cancer, a sarcoma, a skin cancer, a stomach cancer, a testis cancer, a thyroid cancer, a urogenital cancer, or a urothelial cancer; or the cancer is selected from the group consisting of acute myeloid leukemia, adrenocortical carcinoma, B-cell lymphoma, bladder urothelial carcinoma, breast ductal carcinoma, breast lobular carcinoma, carcinomas of the esophagus, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin's lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma.

20. The method of claim 18, wherein the bispecific polypeptide targets at least one of: intratumoral Treg that highly expresses CTLA4, the bispecific polypeptide blockades CTLA4 to allow T cell activation, triggers FcR cell dependent depletion, induces FcγR ADCC (antibody-dependent cellular cytotoxicity) and ADCP (antibody-dependent phagocytosis); or Sirpα binds CD47 deplete Tregs by macrophages; or wherein the Tregs are intratumoral Tregs.

21. A method of depleting or inactivating Tregs comprising contacting an effective amount of a bispecific polypeptide of claim 1 to a T cell and antigen presenting cells that express Sirpα, CD47 or FcR, wherein the antibody induces antibody dependent cell cytotoxicity (ADCC), antibody-dependent cellular phagocytosis (ADCP), complement-dependent cytotoxicity (CDC), and/or apoptosis of the Treg, or wherein the Tregs are intratumoral Tregs.

22. A method of making a regulatory T cell (Treg) depleting protein comprising:
providing an antibody that blocks a T cell inactivating cell surface antigen, wherein the antibody comprises at least one antigen-binding domain and an Fc domain; and
providing a macrophage or dendritic cell surface protein that specifically binds a second T cell target attached to at least one Fc domain polypeptide of the antibody, wherein the polypeptide depletes or inactivates Treg cells.

23. The method of claim 22, wherein the antigen-binding domain binds to CTLA4, OX40, IL2Ra, and CD25; wherein the macrophage or dendritic cell surface protein is Sirpα, CD47, and FcR; wherein the anti-CTLA4 antigen-binding domain is a pH sensitive anti-CTLA4 antibody or fragment thereof; wherein the antigen-binding domain is selected from the group consisting of: an Fv fragment, a single chain Fv fragment, a disulfide-bonded Fv fragment, an Fab fragment; an Fab′ fragment, or an F(ab)₂ fragment); wherein the Fc domain is a human Fc domain or a variant of a said domain, where the Fc domain is an IgG1, IgG2, IgG3 or IgG4 domain, preferably an IgG1 or IgG4 domain; wherein the protein is capable of inducing antibody dependent cell cytotoxicity (ADCC), antibody-dependent cellular phagocytosis (ADCP), complement-dependent cytotoxicity (CDC), and/or apoptosis; wherein the protein is capable of inducing tumor immunity by depleting Treg cells; wherein the Fc domain is a mutant Fc domain that has higher antibody dependent cell cytotoxicity (ADCC), antibody-dependent cellular phagocytosis (ADCP), or complement-dependent cytotoxicity (CDC), when compared to a non-mutant Fc domain; wherein the Fc domain is a human Fc domain or a variant Fc domain, where the Fc domain is an IgG1, IgG2, IgG3 or IgG4, IgA1, IgA2, IgE, IgD or IgM domain; or wherein the polypeptide at least one of: (1) binds to intratumoral Tregs that expresses CTLA4; (2) blockades CTLA4 to allow T cell activation; (3) depletes Tregs in an FcR dependent manner; (4) comprises an FcγR that induces ADCC (antibody-dependent cellular cytotoxicity) and ADCP (antibody-dependent phagocytosis); or (5) wherein the macrophage or dendritic cell surface protein is Sirpα that blocks CD47 and promoted Treg depletion by macrophages.

24. A method of treating, alleviating a symptom of, or delaying a progression of a cancer comprising administering an effective amount of a protein of claim 16 to a subject in need thereof.

25. The method of claim 24, wherein the cancer is selected from a bladder cancer, a bone cancer, a breast cancer, a carcinoid, a cervical cancer, a colon cancer, an endometrial cancer, a glioma, a head and neck cancer, a liver cancer, a lung cancer, a lymphoma, a melanoma, an ovarian cancer, a pancreatic cancer, a prostate cancer, a renal cancer, a sarcoma, a skin cancer, a stomach cancer, a testis cancer, a thyroid cancer, a urogenital cancer, or a urothelial cancer; or wherein the cancer is selected from the group consisting of acute myeloid leukemia, adrenocortical carcinoma, B-cell lymphoma, bladder urothelial carcinoma, breast ductal carcinoma, breast lobular carcinoma, carcinomas of the esophagus, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin's lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung squamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma.

26. The method of claim 24, wherein the protein targets at least one of: intratumoral Treg that highly expresses CTLA4, the bispecific polypeptide blockades CTLA4 to allow T cell activation, triggers FcR cell dependent depletion, induces FcγR ADCC (antibody-dependent cellular cytotoxicity) and ADCP (antibody-dependent phagocytosis); or the Sirpα blocks CD47 deplete Tregs by macrophages; or wherein the Tregs are intratumoral Tregs.

27. A method of depleting or inactivating Tregs comprising contacting an effective amount of a bispecific polypeptide of claim 1 to T cells and antigen presenting cells that express Sirpα, CD47 or FcR, wherein the antibody induces antibody dependent cell cytotoxicity (ADCC), antibody-dependent cellular phagocytosis (ADCP), complement-dependent cytotoxicity (CDC), or apoptosis of the Treg; or wherein the Tregs are intratumoral Tregs.


SEQUENCE LISTING (31 sequences)

SEQ ID NO: 1 - protein, 235 aa, Artificial Sequence
  Synthetic peptide
METDTLLLWVLLLWVPGSTGEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 468 aa, Artificial Sequence
  Synthetic peptide
METDTLLLWVLLLWVPGSTGQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 3 - protein, 377 aa, Artificial Sequence
  Synthetic peptide
METDTLLLWVLLLWVPGSTGMEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 4 - protein, 698 aa, Artificial Sequence
  Synthetic peptide
METDTLLLWVLLLWVPGSTGEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSSGRSENIRTAGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 698 aa, Artificial Sequence; identical to SEQ ID NO: 4
  Synthetic peptide

SEQ ID NO: 6 - protein, 698 aa, Artificial Sequence
  Synthetic peptide
METDTLLLWVLLLWVPGSTGEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSSGRSENIRTAGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 7 - protein, 357 aa, Artificial Sequence
  Synthetic peptide
MEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 8 - protein, 831 aa, Artificial Sequence
  Synthetic peptide
METDTLLLWVLLLWVPGSTGEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRSENIRQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSMEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPS

SEQ ID NO: 9 - protein, 831 aa, Artificial Sequence
  Synthetic peptide
METDTLLLWVLLLWVPGSTGMEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRSENIRQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 1060 aa, Artificial Sequence
  Synthetic peptide
METDTLLLWVLLLWVPGSTGEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSSGRSENIRTAGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGRSENIRTAMEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 235 aa, Artificial Sequence; identical to SEQ ID NO: 1
  Synthetic peptide

SEQ ID NO: 12 - protein, 830 aa, Artificial Sequence
  Synthetic peptide
METDTLLLWVLLLWVPGSTGQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGRSENIRTAMEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 235 aa, Artificial Sequence; identical to SEQ ID NO: 1
  Synthetic peptide

SEQ ID NO: 14 - protein, 468 aa, Artificial Sequence; identical to SEQ ID NO: 2
  Synthetic peptide

SEQ ID NO: 15 - protein, 508 aa, Artificial Sequence
  Synthetic peptide
METDTLLLWVLLLWVPGSTGQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPSGRSENIRTAMEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 16 - protein, 235 aa, Artificial Sequence; identical to SEQ ID NO: 1
  Synthetic peptide

SEQ ID NO: 17 - protein, 724 aa, Artificial Sequence
  Synthetic peptide
METDTLLLWVLLLWVPGSTGQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSMEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSSGRSENIRTAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSP

SEQ ID NO: 18 - protein, 323 aa, Artificial Sequence
  Synthetic peptide
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 19 - protein, 120 aa, Artificial Sequence
  Synthetic peptide
MEEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPS

SEQ ID NO: 20 - protein, 10 aa, Artificial Sequence
  Synthetic peptide
GGGGSGGGGS

SEQ ID NO: 21 - protein, 12 aa, Artificial Sequence
  Synthetic peptide
  MISC_FEATURE (6)..(12): N is any number
GGGGSNGGGGSN

SEQ ID NO: 22 - protein, 6 aa, Artificial Sequence
  Synthetic peptide
  MISC_FEATURE (6)..(6): N is any number
GGGGSN

SEQ ID NO: 23 - protein, 5 aa, Artificial Sequence
  Synthetic peptide
GGGGS

SEQ ID NO: 24 - protein, 10 aa, Artificial Sequence
  Synthetic peptide
GGGGSGGGGS

SEQ ID NO: 25 - protein, 15 aa, Artificial Sequence
  Synthetic peptide
GGGGSGGGGSGGGGS

SEQ ID NO: 26 - protein, 21 aa, Artificial Sequence
  Synthetic peptide
  MISC_FEATURE (11)..(11): X is A or N
GGGGSGGGGSXGGGGSGGGGS

SEQ ID NO: 27 - protein, 21 aa, Artificial Sequence
  Synthetic peptide
  MISC_FEATURE (11)..(11): X is S, A or N
  MISC_FEATURE (16)..(16): Y is A or N
GGGGSGGGGSXGGGGYGGGGS

SEQ ID NO: 28 - protein, 32 aa, Artificial Sequence
  Synthetic peptide
GGGGSGGGGSGGGGSAAGGGGSGGGGSGGGGS

SEQ ID NO: 29 - protein, 6 aa, Artificial Sequence
  Synthetic peptide
PVGLIG

SEQ ID NO: 30 - protein, 10 aa, Artificial Sequence
  Synthetic peptide
SGRSENIRTA

SEQ ID NO: 31 - protein, 10 aa, Artificial Sequence
  Synthetic peptide
SGRSPAIFTA